(19) United States
(12) Patent Application Publication
(10) Pub. No.: US 2010/0216865 A1
(43) Pub. Date: Aug. 26, 2010

(54) MicroRNA COMPOSITIONS IN THE TREATMENT OF VEGF-MEDIATED DISORDERS

(76) Inventor: Jack A. Elias, AICHI (JP)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/677,625

(22) PCT Filed: Sep. 29, 2008
(86) PCT No.: PCT/US08/11242

§ 371 Date: Mar. 11, 2010

(30) Foreign Application Priority Data
Sep. 12, 2007 (JP) 2007-237034

(51) Int. Cl. A61K 31/713; A61P 35/00; A61P 9/10; A61P 29/00; A61P 27/00

(52) U.S. Cl. 514/44 A

(57) ABSTRACT

The invention provides methods of treating diseases caused by the over-production of a VEGF polypeptide by administering miRNA or miRNA inhibitor compositions to decrease at least one activity of a VEGF polypeptide.

### RELATED APPLICATIONS

This application is related to provisional application U.S. Ser. No. 60/995,863, filed Sep. 28, 2007, the contents which are each herein incorporated by reference in their entirety.

### FIELD OF THE INVENTION

This invention relates generally to the fields of cancer, inflammation, fibrotic disease, macular degeneration, and molecular biology.

### BACKGROUND OF THE INVENTION

Vascular endothelial growth factor (VEGF) is a term that encompasses a sub-family of growth factors that have diverse functions in both developing and mature individuals. VEGF is a well-known critical regulator of angiogenesis. For this reason, VEGF has become a target for drug design in cancer among other disorders. However, despite extensive efforts to develop anti-VEGF therapies, a uniformly effective VEGF treatment has not been developed.

### SUMMARY OF THE INVENTION

Methods of the invention provide means for reducing VEGF-induced inflammation, angiogenesis, hemorrhage, endothelial cell proliferation, and prolonged or abortive wound healing by administering miRNA or miRNA inhibitor compositions. Moreover, methods of the invention provide means for treating a disorder caused by the ectopic or overproduction of a vascular endothelial growth factor (VEGF) polypeptide by administering miRNA or miRNA inhibitor compositions to a subject. Compositions of the invention include miRNAs that alter the ability of VEGF to induce cellular and tissue responses or changes.

Specifically, the invention provides a method for treating a disorder caused by the ectopic or overproduction of a vascular endothelial growth factor (VEGF) polypeptide by at least one cell in a subject including administering to the subject an effective amount of an miRNA composition to decrease the amount of a VEGF polypeptide produced by the cell. Alternatively, or in addition, the invention provides a method for treating a disorder caused by the ectopic or overproduction of a vascular endothelial growth factor (VEGF) polypeptide by at least one cell in a subject including administering to the subject an effective amount of an miRNA composition to decrease the ability of VEGF to induce a response by the cell. The invention also provides a method for treating a disorder caused by the ectopic or overproduction of a vascular endothelial growth factor (VEGF) polypeptide by at least one cell in a subject including administering to the subject an effective amount of an miRNA composition to decrease at least one activity of a VEGF polypeptide on the cell. As used herein, the term “activity of a VEGF polypeptide on a cell” is meant to describe the ability of VEGF to induce a response in a cell or tissue.

The invention provides a method for treating a disorder caused by the ectopic or overproduction of a vascular endothelial growth factor (VEGF) polypeptide by at least one cell in a subject including administering to the subject an effective amount of an miRNA inhibitor composition to decrease the amount of a VEGF polypeptide produced by the cell. Alternatively, or in addition, the invention provides a method for treating a disorder caused by the ectopic or overproduction of a vascular endothelial growth factor (VEGF) polypeptide by at least one cell in a subject including administering to the subject an effective amount of an miRNA inhibitor composition to decrease the ability of VEGF to induce a response by the cell. The invention also provides a method for treating a disorder caused by the ectopic or overproduction of a vascular endothelial growth factor (VEGF) polypeptide by at least one cell in a subject including administering to the subject an effective amount of an miRNA inhibitor composition to decrease at least one activity of a VEGF polypeptide on the cell.

The invention provides a method for enhancing wound healing by decreasing production of a vascular endothelial growth factor (VEGF) polypeptide by at least one cell in a subject including administering to the subject an effective amount of an miRNA composition to decrease the amount of a VEGF polypeptide produced by the cell, thereby decreasing the occurrence of VEGF-mediated prolonged or abortive wound healing. Alternatively, or in addition, the invention provides a method for enhancing wound healing by decreasing production of a vascular endothelial growth factor (VEGF) polypeptide by at least one cell in a subject including administering to the subject an effective amount of an miRNA composition to decrease the ability of VEGF to induce a response by the cell, thereby decreasing the occurrence of VEGF-mediated prolonged or abortive wound healing. The invention also provides a method for enhancing wound healing by decreasing production of a vascular endothelial growth factor (VEGF) polypeptide by at least one cell in a subject including administering to the subject an effective amount of an miRNA composition to decrease at least one activity of a VEGF polypeptide on the cell, thereby decreasing the occurrence of VEGF-mediated prolonged or abortive wound healing.

The invention provides a method of decreasing angiogenesis in a tissue, including administering to the tissue an effective amount of an miRNA composition to decrease the amount of a VEGF polypeptide produced by the tissue. Alternatively, or in addition, the invention provides a method of decreasing angiogenesis in a tissue, including administering to the tissue an effective amount of an miRNA composition to decrease the ability of VEGF to induce a response by the tissue. The invention also provides a method of decreasing angiogenesis in a tissue, including administering to the tissue an effective amount of an miRNA composition to decrease at least one activity of a VEGF polypeptide on the tissue. Angiogenesis is defined herein as the growth or remodeling of vascular structures. Angiogenesis can be diagnosed or determined by in vivo and in vitro methods including MRI, angiograms, and histochemistry.

The invention provides a method of decreasing inflammation in a tissue, including administering to the tissue an effective amount of an miRNA composition to decrease the amount of a VEGF polypeptide produced by the tissue. Alternatively, or in addition, the invention provides a method of decreasing inflammation in a tissue, including administering to the tissue an effective amount of an miRNA composition to decrease the ability of VEGF to induce a response by the tissue. The invention also provides a method of decreasing inflammation in a tissue, including administering to the tissue an effective amount of an miRNA composition to decrease at least one activity of a VEGF polypeptide on the tissue. Inflammation is defined herein for the purposes of the invention as any intrusion of an immune cell into the a target tissue which is not part of the immune system. Inflammation can be diagnosed or determined by detection of or accumulation of immune cells within a tissue or fluid sample.

The invention provides a method of decreasing hemorrhage in a tissue, including administering to the tissue an effective amount of an miRNA composition to decrease the amount of a VEGF polypeptide produced by the tissue. Alternatively, or in addition, the invention provides a method of decreasing hemorrhage in a tissue, including administering to the tissue an effective amount of an miRNA composition to decrease the ability of VEGF to induce a response by the tissue. The invention also provides a method of decreasing hemorrhage in a tissue, including administering to the tissue an effective amount of an miRNA composition to decrease at least one activity of a VEGF polypeptide on the tissue. Hemorrhage is defined herein as a loss of blood from the circulatory system. Hemorrhage can be diagnosed or determined by detection of or accumulation of blood within a tissue or fluid sample.

The invention provides a method of decreasing endothelial proliferation in a tissue, including administering to the tissue an effective amount of an miRNA composition to decrease the amount of a VEGF polypeptide produced by the tissue. Alternatively, or in addition, the invention provides a method of decreasing endothelial proliferation in a tissue, including administering to the tissue an effective amount of an miRNA composition to decrease the ability of VEGF to induce a response by the tissue. The invention also provides a method of decreasing endothelial proliferation in a tissue, including administering to the tissue an effective amount of an miRNA composition to decrease at least one activity of a VEGF polypeptide on the tissue.

The invention provides a method of increasing or enhancing wound healing in a tissue, including administering to the tissue an effective amount of an miRNA composition to decrease the amount of a VEGF polypeptide produced by the tissue. Alternatively, or in addition, the invention provides a method of increasing or enhancing wound healing in a tissue, including administering to the tissue an effective amount of an miRNA composition to decrease the ability of VEGF to induce a response by the tissue. The invention provides a method of increasing or enhancing wound healing in a tissue, including administering to the tissue an effective amount of an miRNA composition to decrease at least one activity of a VEGF polypeptide on the tissue.

In one aspect of the above methods, the method further includes determining the amount of a VEGF polypeptide produced. In another aspect of the above methods, the method further includes comparing the amount of a VEGF polypeptide produced prior to administration of the composition to the amount of a VEGF polypeptide produced following administration of the composition, wherein a change in the amount indicates that the subject is treated.

In one aspect of the above methods, the method further includes determining the activity of a VEGF polypeptide. In another aspect of the above methods, the method further includes comparing the activity of a VEGF polypeptide prior to administration of the composition to the activity of a VEGF polypeptide following administration of the composition, wherein a change in the activity indicates that the subject is treated.

As used herein, the term “ability to produce a response” is meant to describe the ability of VEGF to elicit an intracellular signaling cascade in one or more cells by binding to one or more receptors, downstream effectors, or signaling molecules, e.g. targets of miRNA compositions of the invention. Nonlimiting examples of targets of miRNA compositions of the invention include PTK9, KIS, ARF4, MGC26690, SFRS9, ADAR, MTX1, KIAA1160, ACPL2, GNPDA2, NETO2, MMD, PTMAP7, RAB11FIP2, UST, FLJ20273, HPS4, LASP1, TIMP3, SERP1, ANK1B1, TH1L, KIF2, INPP5F, ARHGEF18, SLC16A9, DDX5, CAP1, RABGAP1L, C20orf9, IHRK2, SDC4, H3F3B, LIN7C, RABL2A, FLJ21415, KIAA1340, CHST11, TAGLN2, RNF138, C20orf139, PDCD4, PIP3AP, PREX1, TRM4, NP, TDP1, ANKRD29, TIP120A, SLC25A30, SERPINB5, CDW92, LRRC8, KIAA1194, GCH1, KIAA1295, HIST1H31, LOC63929, MGC27345, CHSY1, TRAPPC3, PGM2, EML4, CT120, CTEN, KIAA1598, MXD4, BLCAP, POGK, AXL, LOC126731, POM121, PLEKHB2, LASS2, FBLN2, ARCN1, XPO6, RABL2B, CLG, TRIM2, SH2D4A, HIST1H3B, PFTK1, PARG1, OSBPL7, ARF3, LZTFL1, DHX15, EPB41L4B, POLR2K, CLCN3, OAT, C2orf3, FLJ20519, ZNF264, TM4SF7, HAND2, ACTR3, ADAR, XRCC6, HNRPU, VARS2, CALR, DHX15, G6PD, CAP1, TPM3, XRCC5, C20orf139, PGM2, KIF2, NETO2, POGK, SERP1, TIP120A, IQGAP1, FOXP1, HDAC4, and RELA. (See also, Lim, L. P. et al. Nature. 2005. 433(17): 769-773). As used herein, the term “targets” is meant to describe any genomic sequence, polynucleotide sequence, polypeptide sequence, homolog or fragment thereof that encodes the named target. MiRNA compositions contact or bind any portion of the targeted molecule.

In an embodiment of the invention the disorder caused by the ectopic or overproduction of a vascular endothelial growth factor (VEGF) polypeptide disorder is a cancer. Cancers of the invention include, but are not limited to, a solid tumor selected from the group consisting of adrenocortical carcinoma, AIDS-related cancers, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, central nervous system lymphoma, cervical cancer, colon cancer, colorectal cancer, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, hypopharyngeal cancer, intraocular melanoma, islet cell tumors (endocrine pancreas), Kaposi Sarcoma, kidney (renal cell) cancer, kidney cancer, laryngeal cancer, lip and oral cavity cancer, liver cancer, non-small cell lung cancer, small cell lung cancer, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine sarcoma, skin cancer (melanoma), merkel cell skin carcinoma, small intestine cancer, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilms Tumor.

In an embodiment of the invention the disorder caused by the ectopic or overproduction of a vascular endothelial growth factor (VEGF) polypeptide disorder is angiogenesis. In one aspect, angiogenesis is vasculogenesis or intussusception.

In an embodiment of the invention the disorder caused by the ectopic or overproduction of a vascular endothelial growth factor (VEGF) polypeptide disorder is a fibrotic disorder. Fibrotic disorders of the invention include, but are not limited to, injection fibrosis, endomyocardial fibrosis, mediastinal fibrosis, myelofibrosis, retroperiotoneal fibrosis, progressive massive fibrosis, nephrogenic systemic fibrosis, interstitial lung disease (ILD), idiopathic pulmonary fibrosis (IPF), scleroderma, radiation-induced pulmonary fibrosis, bleomycin lung, sarcoidosis, silicosis, pulmonary fibrosis, familial pulmonary fibrosis, autoimmune disease, renal graft transplant fibrosis, heart graft transplant fibrosis, liver graft transplant fibrosis, scarring, glomerulonephritis, cirrhosis of the liver, systemic sclerosis, or proliferative vitreoretinopathy.

In an embodiment of the invention the disorder caused by the ectopic or overproduction of a vascular endothelial growth factor (VEGF) polypeptide disorder is wet age-related macular degeneration (wet AMD).

In an embodiment of the invention the disorder caused by the ectopic or overproduction of a vascular endothelial growth factor (VEGF) polypeptide disorder an inflammatory disorder. Inflammatory disorders of the invention include but are not limited to, asthma, interstitial lung disease, chronic bronchitis, eosinophilic bronchitis, eosinophilic pneumonia, pneumonia, atopic dermatitis, atopy, allergic rhinitis, idiopathic pulmonary fibrosis, scleroderma, emphysema, autoimmune diseases, chronic prostatitis, glomerulonephritis, hypersensitivities, inflammatory bowel diseases, pelvic inflammatory disease, reperfusion injury, rheumatoid arthritis, transplant rejection, vasculitis, allergies, myopathies, or chronic obstructive lung disease.

In one aspect of the above methods, the miRNA composition includes miR-1, or any homolog thereof. In another aspect of the above methods, the miRNA composition includes miR-203, or any homolog thereof. In an alternate or additional aspect of the invention, the miRNA composition includes miR-21, or any homolog thereof. In certain aspects of the above methods, the miRNA composition includes miR-468, miR-1, miR-451, miR-706, miR-486, miR-203, miR-494, miR-714, miR-705, miR-21, or any combination or any homolog thereof.

In an embodiment of the above methods, the composition includes a pharmaceutically acceptable carrier. Compositions of the invention are administered systemically. Alternatively, or in addition, compositions are administered locally.

In one aspect of the above methods, the VEGF polypeptide is VEGFA, VEGF-B, VEGF-C, VEGF-D, or PGF. In a particular aspect of the above methods, the VEGF polypeptide is an isoform of VEGFA. In another aspect of the above methods, the VEGF polypeptide is human.

In one aspect of the above methods, cells of the invention include, but are not limited to, mouse lung epithelial cells, mouse lung epithelial cells, and primary pulmonary artery smooth muscle cells.

### BRIEF DESCRIPTION OF THE DRAWINGS

FIG. 1A is a microRNA microarray analysis comparing VEGF₁₆₅ transgene (−) (Sample A) and VEGF₁₆₅ transgene (+) (Sample B) mice.

FIG. 1B is a table showing the signal strength of 10 selected microRNAs in Sample A, Sample B, and the log ratio between the two samples from the microRNA microarray of FIG. 1A.

FIG. 2 is a series of bar graphs illustrating the real time quantitative polymerase chain reaction (qPCR) evaluations of the levels of miRNAs miR-1, miR-451 and miR-203 in lung tissue of VEGF₁₆₅ transgene (−) (wild type, WT) and VEGF₁₆₅ transgene (+) mice (numbers of mice included in each experiment are provided below each bar).

FIG. 3 is a series of bar graphs illustrating the real time quantitative polymerase chain reaction (qPCR) evaluations of the levels of miRNAs miR-1, miR-451 and miR-203 in lung endothelial cells incubated in the presence or absence of VEGF. (**P<0.01)

FIG. 4 is a series of bar graphs illustrating the real time quantitative polymerase chain reaction (qPCR) evaluations of the levels of miRNAs miR-1, miR-451 and miR-203 in pulmonary artery smooth muscle cells incubated in the presence or absence of VEGF.

FIG. 5 is a series of bar graphs illustrating the real time quantitative polymerase chain reaction (qPCR) evaluations of the levels of miRNAs miR-1, miR-451 and miR-203 in lung epithelial cells incubated in the presence or absence of VEGF.

FIG. 6 is a series of photographs (above) and a schematic representation of the treatment scheme (below) showing that a bronchoalveolar lavage (BAL) hemorrhage observed in VEGF₁₆₅ transgene (+) mice is significantly decreased by miR-1 supplementation, but not by supplementation with the siRNA buffer control alone.

FIG. 7 is a series of bar graphs comparing the number of macrophage, lymphocyte, eosinophil, and neutrophil cells within the total BAL cell population collected from VEGF₁₆₅ transgene (−) and VEGF₁₆₅ transgene (+) mice supplemented with miR-1 or a negative control siRNA in control vehicle (buffer) illustrating that miR-1 decreases VEGF-induced inflammation.

FIG. 8 a series of photographs of mouse trachea tissue collected from VEGF165 transgene (−) and VEGF165 transgene (+) mice supplemented with miR-1 or buffer, illustrating that miR-1 abrogates VEGF-induced angiogenesis.

FIG. 9 is a graph showing cell counts of mouse lung endothelial cells (MLECs) in culture following 24-hour exposure to either VEGF or PBS.

FIG. 10A is a graph showing cell counts of mouse lung endothelial cells (MLECs) in culture that have been first transfected with a negative control double stranded RNA (QS) and subsequently exposed to either VEGF or PBS 24-hours post-transfection.

FIG. 10B is a graph showing cell counts of mouse lung endothelial cells (MLECs) in culture that have been first transfected with miR-1 and subsequently exposed to either VEGF or PBS 24-hours post-transfection.

### DETAILED DESCRIPTION

The miRNA compositions and methods provided by the invention are used to reduce VEGF-mediated angiogenesis, inflammation, and endothelial proliferation in a tissue; enhance or increase wound healing in a tissue by decreasing prolonged and abortive wound healing; and treat inflammatory and fibrotic disorders, wet age-related macular degeneration and cancer.

### MicroRNAs.

MicroRNAs (miRNAs) are small, non-coding RNAs. MiRNAs act by inhibiting transcription and/or translation of messenger RNA (mRNA) into protein by binding to their target mRNAs. While not wishing to be bound by theory, miRNAs inhibit mRNA translation by either causing mRNA degradation or inhibiting translation itself.

MiRNAs are single-stranded RNA molecules of about 21-23 nucleotides in length. MiRNAs are encoded by endogenous and exogenous genes that are transcribed from DNA largely by RNA polymerase II, however, miRNA are never translated into polypeptide sequences. As such, miRNA are considered in the art as “non-coding RNA.” The term “endogenous” gene as used herein is meant to encompass all genes that naturally occur within the genome of an individual. The term “exogenous” gene as used herein is meant to encompass all genes that do not naturally occur within the genome of an individual.

While not limited by theory, the present invention includes and is based in part on the understanding that miRNA biogenesis occurs by the following mechanism. MiRNA are processed from primary mRNA transcripts, called “pri-miRNA” by the nuclease Drosha and the double-stranded RNA binding protein DGCR8/Pasha. Once processed, these transcripts form stem-loop structures referred to as “pre-miRNA”. Pre-miRNA are processed one step further by the endonuclease Dicer, which transforms the double-stranded pre-miRNA molecules into the single-stranded mature miRNA and initiates formation of the RNA-induced silencing complex (RISC). One of the two resulting single-stranded complementary miRNA strands, the guide strand, is selected by the argonaute protein of the RISC and incorporated into the RISC, while the other strand, the anti-guide or passenger strand, is degraded. Following integration into the RISC, miRNAs bind target mRNAs and subsequently inhibit translation or transcription.

MiRNAs are complementary to a part or fragment of one or more mRNAs. Moreover, miRNAs do not require absolute sequence complementarity to bind an mRNA, enabling them to regulate a wide range of target transcripts. As used herein, the term “absolute sequence complementarity” is meant to describe a requirement that each nucleotide pair along the length of two sequences, e.g. a miRNA and a target gene or transcript, bind without gaps. It is common that miRNAs bind to their complementary sites with a lesser degree of complementarity. MiRNAs typically bind target sequences with gaps between matched nucleotides. As used herein, the term “complementary” is meant to describe two sequences in which at least 50% of the nucleotides bind from one sequence to the other sequence in trans.

MiRNAs are frequently complementary to the 3′ UTR of the mRNA transcript, however, miRNAs of the invention bind any region of a target mRNA. Alternatively, or in addition, miRNAs target methylation genomic sites which correspond to genes encoding targeted mRNAs. The methylation state of genomic DNA in part determines the accessibility of that DNA to transcription factors. As such, DNA methylation and de-methylation regulate gene silencing and expression, respectively.

MiRNAs of the invention include, but are not limited to those provide below. Moreover, all homologs of the provided miRNAs are contemplated and encompassed by the invention.

| MiRNA | Mature Sequence | SEQ ID NO: |
|---|---|---|
| miR-468 | gucugugugcguquagucaguau | 1 |
| miR-1 | uauguaugaagaaauguaaaggu | 2 |
| miR-451 | aaaccguuaccauuacugaguu | 3 |
| miR-706 | agagaaacccugucucaaaaaa | 4 |
| miR-486 | uccuguacugagcugccccgag | 5 |
| miR-203 | gugaaauguuuaggaccacuag | 6 |
| miR-494 | ugaaacauacacgggaaaccuc | 7 |
| miR-714 | cgacgagggccggucggucgc | 8 |
| miR-705 | ggugggagguggggugggca | 9 |
| miR-21 | uagcuuaucagacugauguuga | 10 |

### MiRNA Modulators

Compositions and methods of the invention include a miRNA, a molecule that augments the levels of a miRNA and/or an inhibitor of a miRNA that modifies or decreases the production of a VEGF polypeptide or the ability of a VEGF polypeptide to induce a response in at least one cell of a subject.

Contemplated miRNA modulators include, but are not limited to, single or double-stranded RNA or DNA polynucleotides, polypeptides, peptide nucleic acids (PNAs), small molecules, ions, polymers, compounds, antibodies, intrabodies, antagomirs or any combination thereof. MiRNA modulators augment or inhibit miRNA expression levels, activity, and/or function. One exemplary miRNA inhibitor is an antagomir. Antagomirs of the invention are chemically engineered oligonucleotides that specifically and effectively silence the expression of one or more miRNA(s). Antagomirs are cholesterol-conjugated single-stranded RNA molecules of about 21-23 nucleotides in length and are complementary to at least one mature target miRNA.

MiRNA inhibitors of the invention repress or silence the expression or function of an endogenous or exogenous miRNA gene by targeting a genomic sequence, precursor sequence, or the miRNA itself and preventing transcription of the gene or causing degradation of the miRNA or its precursor. For example, an inhibitor is an interfering RNA (RNAi), short interfering RNA (siRNA), short hairpin RNA (shRNA), microRNA (miRNA), double-stranded RNA (dsRNA), antisense oligonucleotide (RNA or DNA), morpholino, or peptide nucleic acid (PNA). In one aspect, the inhibitor is a single-stranded RNA, DNA or PNA that binds to the miRNA, creating a dsRNA, DNA/RNA hybrid, or RNA/PNA hybrid, that is subsequently degraded. In an alternate or additional aspect, the inhibitor is a single-stranded RNA, DNA or PNA that binds to the miRNA, which creates a dsRNA, DNA/RNA hybrid, or RNA/PNA hybrid and prevents the miRNA from binding to a target sequence.

In another aspect of the invention, miRNA inhibitors are tagged with sequences or moieties that cause the miRNA to be degraded or sequestered into a cellular compartment or organelle such that the miRNA cannot bind a target sequence. For instance, the miRNA inhibitor is tagged with a secretory signal that causes the miRNA to be expelled from the cell. Alternatively, or in addition, the miRNA inhibitor is tagged with a ubiquitin tag that causes the miRNA to be degraded.

MiRNA modulators decrease the ability of a VEGF polypeptide to induce a response in a cell or tissue. In one aspect of the invention, a miRNA inhibitor further reduces the ability of a miRNA to decrease the ability of a VEGF polypeptide to induce a response in a cell or tissue, for example, in an additive capacity. In another aspect of the invention, a miRNA inhibitor further reduces the ability of a miRNA to decrease the ability of a VEGF polypeptide to induce a response in a cell or tissue, for example, in a synergistic capacity.

### Vascular Endothelial Growth Factor (VEGF)

Compositions and methods of the invention include a miRNA, a molecule that blocks VEGF induced changes in the levels of iRNA and/or an inhibitor of a miRNA that modifies, e.g. increases or decreases, the production of a VEGF polypeptide or the ability of a VEGF polypeptide to induce a response in at least one cell of a subject. Alternatively, or in addition, compositions and methods of the invention include a miRNA, a molecule that blocks VEGF induced changes in the levels of iRNA and/or an inhibitor of a miRNA that modifies, e.g. increases or decreases, the effects of a VEGF polypeptide in at least one cell of a subject.

As used herein, the term “VEGF” encompasses two families of proteins that result from the alternate splicing of a single gene, VEGF, composed of 8 exons. The alternate splice sites reside in the exons 6, 7, and 8. However, the alternate splice site in the terminal exon 8 is functionally important. One family of proteins arises from the proximal splice site and is denoted (VEGFₓₓₓ). Proteins produced by alternate splicing at this proximal location are pro-angiogenic and are expressed conditionally (for instance, when tissues are hypoxic and secreted signals induce angiogenesis). The other family of proteins arises from the distal splice site and is denoted (VEGFₓₓₓb). Proteins produced by alternate splicing at this distal location are anti-angiogenic and are expressed in healthy tissues under normal conditions.

VEGF exons 6 and 7 contain splice sites that result in the inclusion or exclusion of exons 6 and 7, and which affects heparin binding affinity and amino acid number. Heparin binding affinity, interactions with heparin surface proteoglycans (HSPGs) and neuropilin co-receptors on the cell surface mediated by amino acid sequences in exons 6 and 7 enhance the ability of VEGF variants to activate VEGF signaling receptors (VEGFRs).

Endogenous VEGF splice variants are released from cells as glycosylated disulfide-bonded dimers. Structurally, VEGF belongs to the PDGF family of cysteine-knot growth factors including placenta growth factor (PGF), VEGF-B, VEGF-C and VEGF-D. VEGF is sometimes referred to as VEGF-A to differentiate it from these related growth factors.

VEGF-A isoforms mediate angiogenesis, chemotaxis for macrophage and granulocyte cells, and vasodilation. VEGF-B mediates embryonic angiogenesis. VEGF-C signaling is important for lymphangiogenesis. VEGF-D mediates the development of lymphatic vasculature surrounding lung bronchioles. Finally, PGF mediates vasculogenesis and angiogenesis during ischemia, inflammation, wound healing, and cancer progression. Methods of the invention provide miRNAs and inhibitors of miRNAs that target these VEGF family members and/or regulators, either inhibitors/antagonists or activators/agonists, of these family members in any cell type.

Members of the VEGF family stimulate cellular responses by binding to cell-surface tyrosine kinase receptors (the VEGFRs). VEGF-A binds to VEGFR-1 (also known as Flt-1) and VEGFR-2 (also known as KDR/Flk-1). VEGFR-2 is the predominant receptor for VEGF-A mediating almost all of the known cellular responses to this growth factor. The function of VEGFR-1 is unclear, although it is thought to modulate VEGFR-2 signaling. VEGFR-1 may also sequester VEGF from VEGFR-2 binding.

The invention includes all VEGF polynucleotide and polypeptides generated from alternative splicing including pro- and anti-angiogenic forms. Exemplary VEGF polynucleotide and polypeptide splice forms encompassed by the invention include, but are not limited to, the polynucleotides and polypeptides described by the following sequences. Moreover, the invention encompasses all VEGF family members including, but not limited to, VEGF-B, VEGF-C, VEGF-D, and PGF.

Human Vascular Endothelial Growth Factor A (VEGFA), transcript variant 1, is encoded by the following mRNA sequence (NCBI Accession No. NM_{—}001025366 and SEQ ID NO: 11):

```
      1 ggcttggggc agccgggtag ctcggaggtc gtggcgctgg
          gggctagcac cagcgctctg
    61 tcgggaggcg cagcggttag gtggaccggt cagcggactc
          accggccagg gcgctcggtg
  121 ctggaatttg atattcattg atccgggttt tatccctctt
          cttttttctt aaacattttt
  181 ttttaaaact gtattgtttc tcgttttaat ttatttttgc
          ttgccattcc ccacttgaat
  241 cgggccgacg gcttggggag attgctctac ttccccaaat
          cactgtggat tttggaaacc
  301 agcagaaaga ggaaagaggt agcaagagct ccagagagaa
          gtcgaggaag agagagacgg
  361 ggtcagagag agcgcgcggg cgtgcgagca gcgaaagcga
          caggggcaaa gtgagtgacc
  421 tgcttttggg ggtgaccgcc ggagcgcggc gtgagccctc
          ccccttggga tcccgcagct
  481 gaccagtcgc gctgacggac agacagacag acaccgcccc
          cagccccagc taccacctcc
  541 tccccggccg gcggcggaca gtggacgcgg cggcgagccg
          cgggcagggg ccggagcccg
  601 cgcccggagg cggggtggag ggggtcgggg ctcgcggcgt
          cgcactgaaa cttttcgtcc
  661 aacttctggg ctgttctcgc ttcggaggag ccgtggtccg
          cgcgggggaa gccgagccga
  721 gcggagccgc gagaagtgct agctcgggcc gggaggagcc
          gcagccggag gagggggagg
  781 aggaagaaga gaaggaagag gagagggggc cgcagtggcg
          actcggcgct cggaagccgg
  841 gctcatggac gggtgaggcg gcggtgtgcg cagacagtgc
          tccagccgcg cgcgctcccc
  901 aggccctggc ccgggcctcg ggccggggag gaagagtagc
          tcgccgaggc gocgaggaga
  961 gcgggccgcc ccacagcccg agccggagag ggagcgcgag
          ccgcgccggc cccggtcggg
1021 cctccgaaac catgaacttt ctgctgtctt gggtgcattg
          gagccttgcc ttgctgctct
1081 acctccacca tgccaagtgg tcccaggctg cacccatggc
          agaaggagga gggcagaatc
1141 atcacgaagt ggtgaagttc atggatgtct atcagcgcag
          ctactgccat ccaatcgaga
1201 ccctggtgga catcttccag gagtaccctg atgagatcga
          gtacatcttc aagccatcct
1261 gtgtgcccct gatgcgatgc gggggctgct gcaatgacga
          gggcctggag tgtgtgccca
1321 ctgaggagtc caacatcacc atgcagatta tgcggatcaa
          acctcaccaa ggccagcaca
1381 taggagagat gagcttccta cagcacaaca aatgtgaatg
          cagaccaaag aaagatagag
1441 caagacaaga aaaaaaatca gttcgaggaa agggaaaggg
          gcaaaaacga aagcgcaaga
1501 aatcccggta taagtcctgg agcgtgtacg ttggtgcccg
          ctgctgtcta atgccctgga
1561 gcctccctgg cccccatccc tgtgggcctt gctcagagcg
          gagaaagcat ttgtttgtac
1621 aagatccgca gacgtgtaaa tgttcctgca aaaacacaga
          ctcgcgttgc aaggcgaggc
1681 agcttgagtt aaacgaacgt acttgcagat gtgacaagcc
          gaggcggtga gccgggcagg
1741 aggaaggagc ctccctcagg gtttcgggaa ccagatctct
          caccaggaaa gactgataca
1801 gaacgatcga tacagaaacc acgctgccgc caccacacca
          tcaccatcga cagaacagtc
1861 cttaatccag aaacctgaaa tgaaggaaga ggagactctg
          cgcagagcac tttgggtccg
1921 gagggcgaga ctccggcgga agcattcccg ggcgggtgac
          ccagcacggt ccctcttgga
1981 attggattcg ccattttatt tttcttgctg ctaaatcacc
          gagcccggaa gattagagag
2041 ttttatttct gggattcctg tagacacacc cacocacata
          catacattta tatatatata
2101 tattatatat atataaaaat aaatatctct attttatata
          tataaaatat atatattctt
2161 tttttaaatt aacagtgcta atgttattgg tgtcttcact
          ggatgtattt gactgctgtg
2221 gacttgagtt gggaggggaa tgttcccact cagatcctga
          cagggaagag gaggagatga
2281 gagactctgg catgatcttt tttttgtccc acttggtggg
          gccagggtcc tctcccctgc
2341 ccaggaatgt gcaaggccag ggcatggggg caaatatgac
          ccagttttgg gaacaccgac
2401 aaacccagcc ctggcgctga gcctctctac cccaggtcag
          acggacagaa agacagatca
2461 caggtacagg gatgaggaca ccggctctga ccaggagttt
          ggggagcttc aggacattgc
2521 tgtgctttgg ggattccctc cacatgctgc acgcgcatct
          cgcccccagg ggcactgcct
2581 ggaagattca ggagcctggg cggccttcgc ttactctcac
          ctgcttctga gttgcccagg
2641 agaccactgg cagatgtccc ggcgaagaga agagacacat
          tgttggaaga agcagcccat
2701 gacagctccc cttcctggga ctcgccctca tcctcttcct
          gctccccttc ctggggtgca
2761 gcctaaaagg acctatgtcc tcacaccatt gaaaccacta
          gttctgtccc cocaggagac
2821 ctggttgtgt gtgtgtgagt ggttgacctt cctccatccc
          ctggtccttc ccttcccttc
2881 ccgaggcaca gagagacagg gcaggatcca cgtgcccatt
          gtggaggcag agaaaagaga
2941 aagtgtttta tatacggtac ttatttaata tcccttttta
          attagaaatt aaaacagtta
3001 atttaattaa agagtagggt tttttttcag tattcttggt
          taatatttaa tttcaactat
3061 ttatgagatg tatcttttgc tctctcttgc tctcttattt
          gtaccggttt ttgtatataa
3121 aattcatgtt tccaatctct ctctccctga tcggtgacag
          tcactagctt atcttgaaca
3181 gatatttaat tttgctaaca ctcagctctg ccctccccga
          tcccctggct ccccagcaca
3241 cattcctttg aaataaggtt tcaatataca tctacatact
          atatatatat ttggcaactt
3301 gtatttgtgt gtatatatat atatatatgt ttatgtatat
          atgtgattct gataaaatag
3361 acattgctat tctgtttttt atatgtaaaa acaaaacaag
          aaaaaataga gaattctaca
3421 tactaaatct ctctcctttt ttaattttaa tatttgttat
          catttattta ttggtgctac
3481 tgtttatccg taataattgt ggggaaaaga tattaacatc
          acgtctttgt ctctagtgca
3541 gtttttcgag atattccgta gtacatattt atttttaaac
          aacgacaaag aaatacagat
3601 atatcttaaa aaaaaaaaag cattttgtat taaagaattt
          aattctgatc tcaaaaaaaa
3661 aaaaa
```

Human Vascular Endothelial Growth Factor A (VEGFA), transcript variant 1, isoform a (VEGF₂₀₆), is encoded by the following amino acid sequence (NCBI Accession No. NP_{—}001020537.2 and SEQ ID NO: 12):

MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQEKKSVRGKGKGQKRKRKKSRYKSWSVYVGAR
CCLMPWSLPGPHPCGPCSERRKHLFVQDPQTCKCSCKNTDSRCKARQLEL
NERTCRCDKPRR

Human Vascular Endothelial Growth Factor A (VEGFA), transcript variant 2, is encoded by the following mRNA sequence (NCBI Accession No. NM_{—}003376 and SEQ ID NO: 13):

```
      1 ggcttggggc agccgggtag ctcggaggtc gtggcgctgg
          gggctagcac cagcgctctg
    61 tcgggaggcg cagcggttag gtggaccggt cagcggactc
          accggccagg gcgctcggtg
  121 ctggaatttg atattcattg atccgggttt tatccctctt
          cttttttctt aaacattttt
  181 ttttaaaact gtattgtttc tcgttttaat ttatttttgc
          ttgccattcc ccacttgaat
  241 cgggccgacg gcttggggag attgctctac ttccccaaat
          cactgtggat tttggaaacc
  301 agcagaaaga ggaaagaggt agcaagagct ccagagagaa
          gtcgaggaag agagagacgg
  361 ggtcagagag agcgcgcggg cgtgcgagca gcgaaagcga
          caggggcaaa gtgagtgacc
  421 tgcttttggg ggtgaccgcc ggagcgcggc gtgagccctc
          ccccttggga tcccgcagct
  481 gaccagtcgc gctgacggac agacagacag acaccgcccc
          cagccccagc taccacctcc
  541 tccccggccg gcggcggaca gtggacgcgg cggcgagccg
          cgggcagggg ccggagcccg
  601 cgcccggagg cggggtggag ggggtcgggg ctcgcggcgt
          cgcactgaaa cttttcgtcc
  661 aacttctggg ctgttctcgc ttcggaggag ccgtggtccg
          cgcgggggaa gccgagccga
  721 gcggagccgc gagaagtgct agctcgggcc gggaggagcc
          gcagccggag gagggggagg
  781 aggaagaaga gaaggaagag gagagggggc cgcagtggcg
          actcggcgct cggaagccgg
  841 gctcatggac gggtgaggcg gcggtgtgcg cagacagtgc
          tccagccgcg cgcgctcccc
  901 aggccctggc ccgggcctcg ggccggggag gaagagtagc
          tcgccgaggc gccgaggaga
  961 gcgggccgcc ccacagcccg agccggagag ggagcgcgag
          ccgcgccggc cccggtcggg
1021 cctccgaaac catgaacttt ctgctgtctt gggtgcattg
          gagccttgcc ttgctgctct
1081 acctccacca tgccaagtgg tcccaggctg cacccatggc
          agaaggagga gggcagaatc
1141 atcacgaagt ggtgaagttc atggatgtct atcagcgcag
          ctactgccat ccaatcgaga
1201 ccctggtgga catcttccag gagtaccctg atgagatcga
          gtacatcttc aagccatcct
1261 gtgtgcccct gatgcgatgc gggggctgct gcaatgacga
          gggcctggag tgtgtgccca
1321 ctgaggagtc caacatcacc atgcagatta tgcggatcaa
          acctcaccaa ggccagcaca
1381 taggagagat gagcttccta cagcacaaca aatgtgaatg
          cagaccaaag aaagatagag
1441 caagacaaga aaaaaaatca gttcgaggaa agggaaaggg
          gcaaaaacga aagcgcaaga
1501 aatcccggta taagtcctgg agcgttccct gtgggccttg
          ctcagagcgg agaaagcatt
1561 tgtttgtaca agatccgcag acgtgtaaat gttcctgcaa
          aaacacagac tcgcgttgca
1621 aggcgaggca gcttgagtta aacgaacgta cttgcagatg
          tgacaagccg aggcggtgag
1681 ccgggcagga ggaaggagcc tccctcaggg tttcgggaac
          cagatctctc accaggaaag
1741 actgatacag aacgatcgat acagaaacca cgctgccgcc
          accacaccat caccatcgac
1801 agaacagtcc ttaatccaga aacctgaaat gaaggaagag
          gagactctgc gcagagcact
1861 ttgggtccgg agggcgagac tccggcggaa gcattcccgg
          gcgggtgacc cagcacggtc
1921 cctcttggaa ttggattcgc cattttattt ttcttgctgc
          taaatcaccg agcccggaag
1981 attagagagt tttatttctg ggattcctgt agacacaccc
          acccacatac atacatttat
2041 atatatatat attatatata tataaaaata aatatctcta
          ttttatatat ataaaatata
2101 tatattcttt ttttaaatta acagtgctaa tgttattggt
          gtcttcactg gatgtatttg
2161 actgctgtgg acttgagttg ggaggggaat gttcccactc
          agatcctgac agggaagagg
2221 aggagatgag agactctggc atgatctttt ttttgtccca
          cttggtgggg ccagggtcct
2281 ctcccctgcc caggaatgtg caaggccagg gcatgggggc
          aaatatgacc cagttttggg
2341 aacaccgaca aacccagccc tggcgctgag cctctctacc
          ccaggtcaga cggacagaaa
2401 gacagatcac aggtacaggg atgaggacac cggctctgac
          caggagtttg gggagcttca
2461 ggacattgct gtgctttggg gattccctcc acatgctgca
          cgcgcatctc gcccccaggg
2521 gcactgcctg gaagattcag gagcctgggc ggccttcgct
          tactctcacc tgcttctgag
2581 ttgcccagga gaccactggc agatgtcccg gcgaagagaa
          gagacacatt gttggaagaa
2641 gcagcccatg acagctcccc ttcctgggac tcgccctcat
          cctcttcctg ctccccttcc
2701 tggggtgcag cctaaaagga cctatgtcct cacaccattg
          aaaccactag ttctgtcccc
2761 ccaggagacc tggttgtgtg tgtgtgagtg gttgaccttc
          ctccatcccc tggtccttcc
2821 cttcccttcc cgaggcacag agagacaggg caggatccac
          gtgcccattg tggaggcaga
2881 gaaaagagaa agtgttttat atacggtact tatttaatat
          ccctttttaa ttagaaatta
2941 aaacagttaa tttaattaaa gagtagggtt ttttttcagt
          attcttggtt aatatttaat
3001 ttcaactatt tatgagatgt atcttttgct ctctcttgct
          ctcttatttg taccggtttt
3061 tgtatataaa attcatgttt ccaatctctc tctccctgat
          cggtgacagt cactagctta
3121 tcttgaacag atatttaatt ttgctaacac tcagctctgc
          cctccccgat cccctggctc
3181 cccagcacac attcctttga aataaggttt caatatacat
          ctacatacta tatatatatt
3241 tggcaacttg tatttgtgtg tatatatata tatatatgtt
          tatgtatata tgtgattctg
3301 ataaaataga cattgctatt ctgtttttta tatgtaaaaa
          caaaacaaga aaaaatagag
3361 aattctacat actaaatctc tctccttttt taattttaat
          atttgttatc atttatttat
3421 tggtgctact gtttatccgt aataattgtg gggaaaagat
          attaacatca cgtctttgtc
3481 tctagtgcag tttttcgaga tattccgtag tacatattta
          tttttaaaca acgacaaaga
3541 aatacagata tatcttaaaa aaaaaaaagc attttgtatt
          aaagaattta attctgatct
3601 caaaaaaaaa aaaa
```

Human Vascular Endothelial Growth Factor A (VEGFA), transcript variant 2, isoform b (VEGF₁₈₉), is encoded by the following amino acid sequence (NCBI Accession No. NP_{—}003367.4 and SEQ ID NO: 14):

MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGGCNDEGLECVPTEESNITMQIMRIKPHQGQHIGEM
SFLQHNKCECRPKKDRARQEKKSVRGKGKGQKRKRKKSRYKSWSVPCGPC
SERRKHLFVQDPQTCKCSCKNTDSRCKARQLELNERTCRCDKPRR

Human Vascular Endothelial Growth Factor A (VEGFA), transcript variant 3, is encoded by the following mRNA sequence (NCBI Accession No. NM_{—}001025367 and SEQ ID NO: 15):

```
      1 ggcttggggc agccgggtag ctcggaggtc gtggcgctgg
          gggctagcac cagcgctctg
    61 tcgggaggcg cagcggttag gtggaccggt cagcggactc
          accggccagg gcgctcggtg
  121 ctggaatttg atattcattg atccgggttt tatccctctt
          cttttttctt aaacattttt
  181 ttttaaaact gtattgtttc tcgttttaat ttatttttgc
          ttgccattcc ccacttgaat
  241 cgggccgacg gcttggggag attgctctac ttccccaaat
          cactgtggat tttggaaacc
  301 agcagaaaga ggaaagaggt agcaagagct ccagagagaa
          gtcgaggaag agagagacgg
  361 ggtcagagag agcgcgcggg cgtgcgagca gcgaaagcga
          caggggcaaa gtgagtgacc
  421 tgcttttggg ggtgaccgcc ggagcgcggc gtgagccctc
          ccccttggga tcccgcagct
  481 gaccagtcgc gctgacggac agacagacag acaccgcccc
          cagccccagc taccacctcc
  541 tccccggccg gcggcggaca gtggacgcgg cggcgagccg
          cgggcagggg ccggagcccg
  601 cgcccggagg cggggtggag ggggtcgggg ctcgcggcgt
          cgcactgaaa cttttcgtcc
  661 aacttctggg ctgttctcgc ttcggaggag ccgtggtccg
          cgcgggggaa gccgagccga
  721 gcggagccgc gagaagtgct agctcgggcc gggaggagcc
          gcagccggag gagggggagg
  781 aggaagaaga gaaggaagag gagagggggc cgcagtggcg
          actcggcgct cggaagccgg
  841 gctcatggac gggtgaggcg gcggtgtgcg cagacagtgc
          tccagccgcg cgcgctcccc
  901 aggccctggc ccgggcctcg ggccggggag gaagagtagc
          tcgccgaggc gccgaggaga
  961 gcgggccgcc ccacagcccg agccggagag ggagcgcgag
          ccgcgccggc cccggtcggg
1021 cctccgaaac catgaacttt ctgctgtctt gggtgcattg
          gagccttgcc ttgctgctct
1081 acctccacca tgccaagtgg tcccaggctg cacccatggc
          agaaggagga gggcagaatc
1141 atcacgaagt ggtgaagttc atggatgtct atcagcgcag
          ctactgccat ccaatcgaga
1201 ccctggtgga catcttccag gagtaccctg atgagatcga
          gtacatcttc aagccatcct
1261 gtgtgcccct gatgcgatgc gggggctgct gcaatgacga
          gggcctggag tgtgtgccca
1321 ctgaggagtc caacatcacc atgcagatta tgcggatcaa
          acctcaccaa ggccagcaca
1381 taggagagat gagcttccta cagcacaaca aatgtgaatg
          cagaccaaag aaagatagag
1441 caagacaaga aaaaaaatca gttcgaggaa agggaaaggg
          gcaaaaacga aagcgcaaga
1501 aatcccgtcc ctgtgggcct tgctcagagc ggagaaagca
          tttgtttgta caagatccgc
1561 agacgtgtaa atgttcctgc aaaaacacag actcgcgttg
          caaggcgagg cagcttgagt
1621 taaacgaacg tacttgcaga tgtgacaagc cgaggcggtg
          agccgggcag gaggaaggag
1681 cctccctcag ggtttcggga accagatctc tcaccaggaa
          agactgatac agaacgatcg
1741 atacagaaac cacgctgccg ccaccacacc atcaccatcg
          acagaacagt ccttaatcca
1801 gaaacctgaa atgaaggaag aggagactct gcgcagagca
          ctttgggtcc ggagggcgag
1861 actccggcgg aagcattccc gggcgggtga cccagcacgg
          tccctcttgg aattggattc
1921 gccattttat ttttcttgct gctaaatcac cgagcccgga
          agattagaga gttttatttc
1981 tgggattcct gtagacacac ccacccacat acatacattt
          atatatatat atattatata
2041 tatataaaaa taaatatctc tattttatat atataaaata
          tatatattct ttttttaaat
2101 taacagtgct aatgttattg gtgtcttcac tggatgtatt
          tgactgctgt ggacttgagt
2161 tgggagggga atgttcccac tcagatcctg acagggaaga
          ggaggagatg agagactctg
2221 gcatgatctt ttttttgtcc cacttggtgg ggccagggtc
          ctctcccctg cccaggaatg
2281 tgcaaggcca gggcatgggg gcaaatatga cccagttttg
          ggaacaccga caaacccagc
2341 cctggcgctg agcctctcta ccccaggtca gacggacaga
          aagacagatc acaggtacag
2401 ggatgaggac accggctctg accaggagtt tggggagctt
          caggacattg ctgtgctttg
2461 gggattccct ccacatgctg cacgcgcatc tcgcccccag
          gggcactgcc tggaagattc
2521 aggagcctgg gcggccttcg cttactctca cctgcttctg
          agttgcccag gagaccactg
2581 gcagatgtcc cggcgaagag aagagacaca ttgttggaag
          aagcagccca tgacagctcc
2641 ccttcctggg actcgccctc atcctcttcc tgctcccctt
          cctggggtgc agcctaaaag
2701 gacctatgtc ctcacaccat tgaaaccact agttctgtcc
          ccccaggaga cctggttgtg
2761 tgtgtgtgag tggttgacct tcctccatcc cctggtcctt
          cccttccctt cccgaggcac
2821 agagagacag ggcaggatcc acgtgcccat tgtggaggca
          gagaaaagag aaagtgtttt
2881 atatacggta cttatttaat atcccttttt aattagaaat
          taaaacagtt aatttaatta
2941 aagagtaggg ttttttttca gtattcttgg ttaatattta
          atttcaacta tttatgagat
3001 gtatcttttg ctctctcttg ctctcttatt tgtaccggtt
          tttgtatata aaattcatgt
3061 ttccaatctc tctctccctg atcggtgaca gtcactagct
          tatcttgaac agatatttaa
3121 ttttgctaac actcagctct gccctccccg atcccctggc
          tccccagcac acattccttt
3181 gaaataaggt ttcaatatac atctacatac tatatatata
          tttggcaact tgtatttgtg
3241 tgtatatata tatatatatg tttatgtata tatgtgattc
          tgataaaata gacattgcta
3301 ttctgttttt tatatgtaaa aacaaaacaa gaaaaaatag
          agaattctac atactaaatc
3361 tctctccttt tttaatttta atatttgtta tcatttattt
          attggtgcta ctgtttatcc
3421 gtaataattg tggggaaaag atattaacat cacgtctttg
          tctctagtgc agtttttcga
3481 gatattccgt agtacatatt tatttttaaa caacgacaaa
          gaaatacaga tatatcttaa
3541 aaaaaaaaaa gcattttgta ttaaagaatt taattctgat
          ctcaaaaaaa aaaaaa
```

Human Vascular Endothelial Growth Factor A (VEGFA), transcript variant 3, isoform c (VEGF₁₈₃), is encoded by the following amino acid sequence (NCBI Accession No. NP_{—}001020538.2 and SEQ ID NO: 36):

MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQEKKSVRGKGKGQKRKRKKSRPcGPCSERRKH
LFVQDPQTCKCSCKNTDSRCKARQLELNERTCRCDKPRR

Human Vascular Endothelial Growth Factor A (VEGFA), transcript variant 4, is encoded by the following mRNA sequence (NCBI Accession No. NM_{—}001025368 and SEQ ID NO: 16):

```
      1 ggcttggggc agccgggtag ctcggaggtc gtggcgctgg
          gggctagcac cagcgctctg
    61 tcgggaggcg cagcggttag gtggaccggt cagcggactc
          accggccagg gcgctcggtg
  121 ctggaatttg atattcattg atccgggttt tatccctctt
          cttttttctt aaacattttt
  181 ttttaaaact gtattgtttc tcgttttaat ttatttttgc
          ttgccattcc ccacttgaat
  241 cgggccgacg gcttggggag attgctctac ttccccaaat
          cactgtggat tttggaaacc
  301 agcagaaaga ggaaagaggt agcaagagct ccagagagaa
          gtcgaggaag agagagacgg
  361 ggtcagagag agcgcgcggg cgtgcgagca gcgaaagcga
          caggggcaaa gtgagtgacc
  421 tgcttttggg ggtgaccgcc ggagcgcggc gtgagccctc
          ccccttggga tcccgcagct
  481 gaccagtcgc gctgacggac agacagacag acaccgcccc
          cagccccagc taccacctcc
  541 tccccggccg gcggcggaca gtggacgcgg cggcgagccg
          cgggcagggg ccggagcccg
  601 cgcccggagg cggggtggag ggggtcgggg ctcgcggcgt
          cgcactgaaa cttttcgtcc
  661 aacttctggg ctgttctcgc ttcggaggag ccgtggtccg
          cgcgggggaa gccgagccga
  721 gcggagccgc gagaagtgct agctcgggcc gggaggagcc
          gcagccggag gagggggagg
  781 aggaagaaga gaaggaagag gagagggggc cgcagtggcg
          actcggcgct cggaagccgg
  841 gctcatggac gggtgaggcg gcggtgtgcg cagacagtgc
          tccagccgcg cgcgctcccc
  901 aggccctggc ccgggcctcg ggccggggag gaagagtagc
          tcgccgaggc gccgaggaga
  961 gcgggccgcc ccacagcccg agccggagag ggagcgcgag
          ccgcgccggc cccggtcggg
1021 cctccgaaac catgaacttt ctgctgtctt gggtgcattg
          gagccttgcc ttgctgctct
1081 acctccacca tgccaagtgg tcccaggctg cacccatggc
          agaaggagga gggcagaatc
1141 atcacgaagt ggtgaagttc atggatgtct atcagcgcag
          ctactgccat ccaatcgaga
1201 ccctggtgga catcttccag gagtaccctg atgagatcga
          gtacatcttc aagccatcct
1261 gtgtgcccct gatgcgatgc gggggctgct gcaatgacga
          gggcctggag tgtgtgccca
1321 ctgaggagtc caacatcacc atgcagatta tgcggatcaa
          acctcaccaa ggccagcaca
1381 taggagagat gagcttccta cagcacaaca aatgtgaatg
          cagaccaaag aaagatagag
1441 caagacaaga aaatccctgt gggccttgct cagagcggag
          aaagcatttg tttgtacaag
1501 atccgcagac gtgtaaatgt tcctgcaaaa acacagactc
          gcgttgcaag gcgaggcagc
1561 ttgagttaaa cgaacgtact tgcagatgtg acaagccgag
          gcggtgagcc gggCaggagg
1621 aaggagcctc cctcagggtt tcgggaacca gatctctcac
          caggaaagac tgatacagaa
1681 cgatcgatac agaaaccacg ctgccgccac cacaccatca
          ccatcgacag aacagtcctt
1741 aatccagaaa cctgaaatga aggaagagga gactctgcgc
          agagcacttt gggtccggag
1801 ggcgagactc cggcggaagc attcccgggc gggtgaccca
          gcacggtccc tcttggaatt
1861 ggattcgcca ttttattttt cttgctgcta aatcaccgag
          cccggaagat tagagagttt
1921 tatttctggg attcctgtag acacacccac ccacatacat
          acatttatat atatatatat
1981 tatatatata taaaaataaa tatctctatt ttatatatat
          aaaatatata tattcttttt
2041 ttaaattaac agtgctaatg ttattggtgt cttcactgga
          tgtatttgac tgctgtggac
2101 ttgagttggg aggggaatgt tcccactcag atcctgacag
          ggaagaggag gagatgagag
2161 actctggcat gatctttttt ttgtcccact tggtggggcc
          agggtcctct cccctgccca
2221 ggaatgtgca aggccagggc atgggggcaa atatgaccca
          gttttgggaa caccgacaaa
2281 cccagccctg gcgctgagcc tctctacccc aggtcagacg
          gacagaaaga cagatcacag
2341 gtacagggat gaggacaccg gctctgacca ggagtttggg
          gagcttcagg acattgctgt
2401 gctttgggga ttccctccac atgctgcacg cgcatctcgc
          ccccaggggc actgcctgga
2461 agattcagga gcctgggcgg ccttcgctta ctctcacctg
          cttctgagtt gcccaggaga
2521 ccactggcag atgtcccggc gaagagaaga gacacattgt
          tggaagaagc agcccatgac
2581 agctcccctt cctgggactc gccctcatcc tcttcctgct
          ccccttcctg gggtgcagcc
2641 taaaaggacc tatgtcctca caccattgaa accactagtt
          ctgtcccccc aggagacctg
2701 gttgtgtgtg tgtgagtggt tgaccttcct ccatcccctg
          gtccttccct tcccttcccg
2761 aggoacagag agacagggca ggatccacgt gcccattgtg
          gaggcagaga aaagagaaag
2821 tgttttatat acggtactta tttaatatcc ctttttaatt
          agaaattaaa acagttaatt
2881 taattaaaga gtagggtttt ttttcagtat tcttggttaa
          tatttaattt caactattta
2941 tgagatgtat cttttgctct ctcttgctct cttatttgta
          ccggtttttg tatataaaat
3001 tcatgtttcc aatctctctc tccctgatcg gtgacagtca
          ctagcttatc ttgaacagat
3061 atttaatttt gctaacactc agctctgccc tccccgatcc
          cctggctccc cagcacacat
3121 tcctttgaaa taaggtttca atatacatct acatactata
          tatatatttg gcaacttgta
3181 tttgtgtgta tatatatata tatatgttta tgtatatatg
          tgattctgat aaaatagaca
3241 ttgctattct gttttttata tgtaaaaaca aaacaagaaa
          aaatagagaa ttctacatac
3301 taaatctctc tcctttttta attttaatat ttgttatcat
          ttatttattg gtgctactgt
3361 ttatccgtaa taattgtggg gaaaagatat taacatcacg
          tctttgtctc tagtgcagtt
3421 tttcgagata ttccgtagta catatttatt tttaaacaac
          gacaaagaaa tacagatata
3481 tcttaaaaaa aaaaaagcat tttgtattaa agaatttaat
          tctgatctca aaaaaaaaaa
3541 aa
```

Human Vascular Endothelial Growth Factor A (VEGFA), transcript variant 4, isoform d (VEGF₁₆₅), is encoded by the following amino acid sequence (NCBI Accession No. NP_{—}001020539.2 and SEQ ID NO: 17)

MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQENPCGPCSERRKHLFVQDPQTCKCSCKNTDS
RCKARQLELNERTCRCDKPRR

Human Vascular Endothelial Growth Factor A (VEGFA), transcript variant 5, is encoded by the following mRNA sequence (NCBI Accession No. NM_{—}001025369 and SEQ ID NO: 18):

| | |
|---|---|
| 1 | ggcttggggc agccgggtag ctcggaggtc gtggcgctgg gggctagcac cagcgctctg |
| 61 | tcgggaggcg cagcggttag gtggaccggt cagcggactc accggccagg gcgctcggtg |
| 121 | ctggaatttg atattcattg atccgggttt tatccctctt cttttttctt aaacattttt |
| 181 | ttttaaaact gtattgtttc tcgttttaat ttatttttgc ttgccattcc ccacttgaat |
| 241 | cgggccgacg gcttggggag attgctctac ttccccaaat cactgtggat tttggaaacc |
| 301 | agcagaaaga ggaaagaggt agcaagagct ccagagagaa gtcgaggaag agagagacgg |
| 361 | ggtcagagag agcgcgcggg cgtgcgagca gcgaaagcga caggggcaaa gtgagtgacc |
| 421 | tgcttttggg ggtgaccgcc ggagcgcggc gtgagccctc ccccttggga tcccgcagct |
| 481 | gaccagtcgc gctgacggac agacagacag acaccgcccc cagccccagc taccacctcc |
| 541 | tccccggccg gcggcggaca gtggacgcgg cggcgagccg cgggcagggg ccggagcccg |
| 601 | cgcccggagg cggggtggag ggggtcgggg ctcgcggcgt cgcactgaaa cttttcgtcc |
| 661 | aacttctggg ctgttctcgc ttcggaggag ccgtggtccg cgcgggggaa gccgagccga |
| 721 | gcggagccgc gagaagtgct agctcgggcc gggaggagcc gcagccggag gagggggagg |
| 781 | aggaagaaga gaaggaagag gagagggggc cgcagtggcg actcggcgct cggaagccgg |
| 841 | gctcatggac gggtgaggcg gcggtgtgcg cagacagtgc tccagccgcg cgcgctcccc |
| 901 | aggccctggc ccgggcctcg ggccggggag gaagagtagc tcgccgaggc gccgaggaga |
| 961 | gcgggccgcc ccacagcccg agccggagag ggagcgcgag ccgcgccggc cccggtcggg |
| 1021 | cctccgaaac catgaacttt ctgctgtctt gggtgcattg gagccttgcc ttgctgctct |
| 1081 | acctccacca tgccaagtgg tcccaggctg cacccatggc agaaggagga gggcagaatc |
| 1141 | atcacgaagt ggtgaagttc atggatgtct atcagcgcag ctactgccat ccaatcgaga |
| 1201 | ccctggtgga catcttccag gagtaccctg atgagatcga gtacatcttc aagccatcct |
| 1261 | gtgtgcccct gatgcgatgc gggggctgct gcaatgacga gggcctggag tgtgtgccca |
| 1321 | ctgaggagtc caacatcacc atgcagatta tgcggatcaa acctcaccaa ggccagcaca |
| 1381 | taggagagat gagcttccta cagcacaaca aatgtgaatg cagaccaaag aaagatagag |
| 1441 | caagacaaga aaatccctgt gggccttgct cagagcggag aaagcatttg tttgtacaag |
| 1501 | atccgcagac gtgtaaatgt tcctgcaaaa acacagactc gcgttgcaag atgtgacaag |
| 1561 | ccgaggcggt gagccgggca ggaggaagga gcctccctca gggtttcggg aaccagatct |
| 1621 | ctcaccagga aagactgata cagaacgatc gatacagaaa ccacgctgcc gccaccacac |
| 1681 | catcaccatc gacagaacag tccttaatcc agaaacctga aatgaaggaa gaggagactc |
| 1741 | tgcgcagagc actttgggtc cggagggcga gactccggcg gaagcattcc cgggcgggtg |
| 1801 | acccagcacg gtccctcttg gaattggatt cgccatttta tttttcttgc tgctaaatca |
| 1861 | ccgagcccgg aagattagag agttttattt ctgggattcc tgtagacaca cccacccaca |
| 1921 | tacatacatt tatatatata tatattatat atatataaaa ataaatatct ctattttata |
| 1981 | tatataaaat atatatattc tttttttaaa ttaacagtgc taatgttatt ggtgtcttca |
| 2041 | ctggatgtat ttgactgctg tggacttgag ttgggagggg aatgttccca ctcagatcct |
| 2101 | gacagggaag aggaggagat gagagactct ggcatgatct tttttttgtc ccacttggtg |
| 2161 | gggccagggt cctctcccct gcccaggaat gtgcaaggcc agggcatggg ggcaaatatg |
| 2221 | acccagtttt gggaacaccg acaaacccag ccctggcgct gagcctctct accccaggtc |
| 2281 | agacggacag aaagacagat cacaggtaca gggatgagga caccggctct gaccaggagt |
| 2341 | ttggggagct tcaggacatt gctgtgcttt ggggattccc tccacatgct gcacgcgcat |
| 2401 | ctcgccccca ggggcactgc ctggaagatt caggagcctg ggcggccttc gcttactctc |
| 2461 | acctgcttct gagttgccca ggagaccact ggcagatgtc ccggcgaaga gaagagacac |
| 2521 | attgttggaa gaagcagccc atgacagctc cccttcctgg gactcgccct catcctcttc |
| 2581 | ctgctcccct tcctggggtg cagcctaaaa ggacctatgt cctcacacca ttgaaaccac |
| 2641 | tagttctgtc cccccaggag acctggttgt gtgtgtgtga gtggttgacc ttcctccatc |
| 2701 | ccctggtcct tcccttccct tcccgaggca cagagagaca gggcaggatc cacgtgccca |
| 2761 | ttgtggaggc agagaaaaga gaaagtgttt tatatacggt acttatttaa tatccctttt |
| 2821 | taattagaaa ttaaaacagt taatttaatt aaagagtagg gttttttttc agtattcttg |
| 2881 | gttaatattt aatttcaact atttatgaga tgtatctttt gctctctctt gctctcttat |
| 2941 | ttgtaccggt ttttgtatat aaaattcatg tttccaatct ctctctccct gatcggtgac |
| 3001 | agtcactagc ttatcttgaa cagatattta attttgctaa cactcagctc tgccctcccc |
| 3061 | gatcccctgg ctccccagca cacattcctt tgaaataagg tttcaatata catctacata |
| 3121 | ctatatatat atttggcaac ttgtatttgt gtgtatatat atatatatat gtttatgtat |
| 3181 | atatgtgatt ctgataaaat agacattgct attctgtttt ttatatgtaa aaacaaaaca |
| 3241 | agaaaaaata gagaattcta catactaaat ctctctcctt ttttaatttt aatatttgtt |
| 3301 | atcatttatt tattggtgct actgtttatc cgtaataatt gtggggaaaa gatattaaca |
| 3361 | tcacgtcttt gtctctagtg cagtttttcg agatattccg tagtacatat ttatttttaa |
| 3421 | acaacgacaa agaaatacag atatatctta aaaaaaaaaa agcattttgt attaaagaat |
| 3481 | ttaattctga tctcaaaaaa aaaaaaa |

Human Vascular Endothelial Growth Factor A (VEGFA), transcript variant 5, isoform e (VEGF₁₄₈), is encoded by the following amino acid sequence (NCBI Accession No. NP_{—}001020540.2 and SEQ ID NO: 19):

MTDRQTDTAPSPSYHLLPGRRRTVDAAASRGQGPEPAPGGGVEGVGARGV
ALKLFVQLLGGSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQENPCGPCSERRKHLFVQDPQTCKCSCKNTDS
RCKM

Human Vascular Endothelial Growth Factor A (VEGFA), transcript variant 6, is encoded by the following mRNA sequence (NCBI Accession No. NM_{—}001025370 and SEQ ID NO: 20):

| | |
|---|---|
| 1 | ggcttggggc agccgggtag ctcggaggtc gtggcgctgg gggctagcac cagcgctctg |
| 61 | tcgggaggcg cagcggttag gtggaccggt cagcggactc accggccagg gcgctcggtg |
| 121 | ctggaatttg atattcattg atccgggttt tatccctctt cttttttctt aaacattttt |
| 181 | ttttaaaact gtattgtttc tcgttttaat ttatttttgc ttgccattcc ccacttgaat |
| 241 | cgggccgacg gcttggggag attgctctac ttccccaaat cactgtggat tttggaaacc |
| 301 | agcagaaaga ggaaagaggt agcaagagct ccagagagaa gtcgaggaag agagagacgg |
| 361 | ggtcagagag agcgcgcggg cgtgcgagca gcgaaagcga caggggcaaa gtgagtgacc |
| 421 | tgcttttggg ggtgaccgcc ggagcgcggc gtgagccctc ccccttggga tcccgcagct |
| 481 | gaccagtcgc gctgacggac agacagacag acaccgcccc cagccccagc taccacctcc |
| 541 | tccccggccg gcggcggaca gtggacgcgg cggcgagccg cgggcagggg ccggagcccg |
| 601 | cgcccggagg cggggtggag ggggtcgggg ctcgcggcgt cgcactgaaa cttttcgtcc |
| 661 | aacttctggg ctgttctcgc ttcggaggag ccgtggtccg cgcgggggaa gccgagccga |
| 721 | gcggagccgc gagaagtgct agctcgggcc gggaggagcc gcagccggag gagggggagg |
| 781 | aggaagaaga gaaggaagag gagagggggc cgcagtggcg actcggcgct cggaagccgg |
| 841 | gctcatggac gggtgaggcg gcggtgtgcg cagacagtgc tccagccgcg cgcgctcccc |
| 901 | aggccctggc ccgggcctcg ggccggggag gaagagtagc tcgccgaggc gccgaggaga |
| 961 | gcgggccgcc ccacagcccg agccggagag ggagcgcgag ccgcgccggc cccggtcggg |
| 1021 | cctccgaaac catgaacttt ctgctgtctt gggtgcattg gagccttgcc ttgctgctct |
| 1081 | acctccacca tgccaagtgg tcccaggctg cacccatggc agaaggagga gggcagaatc |
| 1141 | atcacgaagt ggtgaagttc atggatgtct atcagcgcag ctactgccat ccaatcgaga |
| 1201 | ccctggtgga catcttccag gagtaccctg atgagatcga gtacatcttc aagccatcct |
| 1261 | gtgtgcccct gatgcgatgc gggggctgct gcaatgacga gggcctggag tgtgtgccca |
| 1321 | ctgaggagtc caacatcacc atgcagatta tgcggatcaa acctcaccaa ggccagcaca |
| 1381 | taggagagat gagcttccta cagcacaaca aatgtgaatg cagaccaaag aaagatagag |
| 1441 | caagacaaga aaaatgtgac aagccgaggc ggtgagccgg gcaggaggaa ggagcctccc |
| 1501 | tcagggtttc gggaaccaga tctctcacca ggaaagactg atacagaacg atcgatacag |
| 1561 | aaaccacgct gccgccacca caccatcacc atcgacagaa cagtccttaa tccagaaacc |
| 1621 | tgaaatgaag gaagaggaga ctctgcgcag agcactttgg gtccggaggg cgagactccg |
| 1681 | gcggaagcat tcccgggcgg gtgacccagc acggtccctc ttggaattgg attcgccatt |
| 1741 | ttatttttct tgctgctaaa tcaccgagcc cggaagatta gagagtttta tttctgggat |
| 1801 | tcctgtagac acacccaccc acatacatac atttatatat atatatatta tatatatata |
| 1861 | aaaataaata tctctatttt atatatataa aatatatata ttcttttttt aaattaacag |
| 1921 | tgctaatgtt attggtgtct tcactggatg tatttgactg ctgtggactt gagttgggag |
| 1981 | gggaatgttc ccactcagat cctgacaggg aagaggagga gatgagagac tctggcatga |
| 2041 | tctttttttt gtcccacttg gtggggccag ggtcctctcc cctgcccagg aatgtgcaag |
| 2101 | gccagggcat gggggcaaat atgacccagt tttgggaaca ccgacaaacc cagccctggc |
| 2161 | gctgagcctc tctaccccag gtcagacgga cagaaagaca gatcacaggt acagggatga |
| 2221 | ggacaccggc tctgaccagg agtttgggga gcttcaggac attgctgtgc tttggggatt |
| 2281 | ccctccacat gctgcacgcg catctcgccc ccaggggcac tgcctggaag attcaggagc |
| 2341 | ctgggcggcc ttcgcttact ctcacctgct tctgagttgc ccaggagacc actggcagat |
| 2401 | gtcccggcga agagaagaga cacattgttg gaagaagcag cccatgacag ctccccttcc |
| 2461 | tgggactcgc cctcatcctc ttcctgctcc ccttcctggg gtgcagccta aaaggaccta |
| 2521 | tgtcctcaca ccattgaaac cactagttct gtccccccag gagacctggt tgtgtgtgtg |
| 2581 | tgagtggttg accttcctcc atcccctggt ccttcccttc ccttcccgag gcacagagag |
| 2641 | acagggcagg atccacgtgc ccattgtgga ggcagagaaa agagaaagtg ttttatatac |
| 2701 | ggtacttatt taatatccct ttttaattag aaattaaaac agttaattta attaaagagt |
| 2761 | agggtttttt ttcagtattc ttggttaata tttaatttca actatttatg agatgtatct |
| 2821 | tttgctctct cttgctctct tatttgtacc ggtttttgta tataaaattc atgtttccaa |
| 2881 | tctctctctc cctgatcggt gacagtcact agcttatctt gaacagatat ttaattttgc |
| 2941 | taacactcag ctctgccctc cccgatcccc tggctcccca gcacacattc ctttgaaata |
| 3001 | aggtttcaat atacatctac atactatata tatatttggc aacttgtatt tgtgtgtata |
| 3061 | tatatatata tatgtttatg tatatatgtg attctgataa aatagacatt gctattctgt |
| 3121 | tttttatatg taaaaacaaa acaagaaaaa atagagaatt ctacatacta aatctctctc |
| 3181 | cttttttaat tttaatattt gttatcattt atttattggt gctactgttt atccgtaata |
| 3241 | attgtgggga aaagatatta acatcacgtc tttgtctcta gtgcagtttt tcgagatatt |
| 3301 | ccgtagtaca tatttatttt taaacaacga caaagaaata cagatatatc ttaaaaaaaa |
| 3361 | aaaagcattt tgtattaaag aatttaattc tgatctcaaa aaaaaaaaaa |

Human Vascular Endothelial Growth Factor A (VEGFA), transcript variant 6, isoform f (VEGF₁₂₁), is encoded by the following amino acid sequence (NCBI Accession No. NP_{—}001020541.2 and SEQ ID NO: 21):

MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQEKCDKPRR

Human Vascular Endothelial Growth Factor A (VEGFA), transcript variant 7, is encoded by the following mRNA sequence (NCBI Accession No. NM_{—}001033756 and SEQ ID NO: 22):

| | |
|---|---|
| 1 | ggcttggggc agccgggtag ctcggaggtc gtggcgctgg gggctagcac cagcgctctg |
| 61 | tcgggaggcg cagcggttag gtggaccggt cagcggactc accggccagg gcgctcggtg |
| 121 | ctggaatttg atattcattg atccgggttt tatccctctt cttttttctt aaacattttt |
| 181 | ttttaaaact gtattgtttc tcgttttaat ttatttttgc ttgccattcc ccacttgaat |
| 241 | cgggccgacg gcttggggag attgctctac ttccccaaat cactgtggat tttggaaacc |
| 301 | agcagaaaga ggaaagaggt agcaagagct ccagagagaa gtcgaggaag agagagacgg |
| 361 | ggtcagagag agcgcgcggg cgtgcgagca gcgaaagcga caggggcaaa gtgagtgacc |
| 421 | tgcttttggg ggtgaccgcc ggagcgcggc gtgagccctc ccccttggga tcccgcagct |
| 481 | gaccagtcgc gctgacggac agacagacag acaccgcccc cagccccagc taccacctcc |
| 541 | tccccggccg gcggcggaca gtggacgcgg cggcgagccg cgggcagggg ccggagcccg |
| 601 | cgcccggagg cggggtggag ggggtcgggg ctcgcggcgt cgcactgaaa cttttcgtcc |
| 661 | aacttctggg ctgttctcgc ttcggaggag ccgtggtccg cgcgggggaa gccgagccga |
| 721 | gcggagccgc gagaagtgct agctcgggcc gggaggagcc gcagccggag gagggggagg |
| 781 | aggaagaaga gaaggaagag gagagggggc cgcagtggcg actcggcgct cggaagccgg |
| 841 | gctcatggac gggtgaggcg gcggtgtgcg cagacagtgc tccagccgcg cgcgctcccc |
| 901 | aggccctggc ccgggcctcg ggccggggag gaagagtagc tcgccgaggc gccgaggaga |
| 961 | gcgggccgcc ccacagcccg agccggagag ggagcgcgag ccgcgccggc cccggtcggg |
| 1021 | cctccgaaac catgaacttt ctgctgtctt gggtgcattg gagccttgcc ttgctgctct |
| 1081 | acctccacca tgccaagtgg tcccaggctg cacccatggc agaaggagga gggcagaatc |
| 1141 | atcacgaagt ggtgaagttc atggatgtct atcagcgcag ctactgccat ccaatcgaga |
| 1201 | ccctggtgga catcttccag gagtaccctg atgagatcga gtacatcttc aagccatcct |
| 1261 | gtgtgcccct gatgcgatgc gggggctgct gcaatgacga gggcctggag tgtgtgccca |
| 1321 | ctgaggagtc caacatcacc atgcagatta tgcggatcaa acctcaccaa ggccagcaca |
| 1381 | taggagagat gagcttccta cagcacaaca aatgtgaatg cagaccaaag aaagatagag |
| 1441 | caagacaaga aaatccctgt gggccttgct cagagcggag aaagcatttg tttgtacaag |
| 1501 | atccgcagac gtgtaaatgt tcctgcaaaa acacagactc gcgttgcaag gcgaggcagc |
| 1561 | ttgagttaaa cgaacgtact tgcagatctc tcaccaggaa agactgatac agaacgatcg |
| 1621 | atacagaaac cacgctgccg ccaccacacc atcaccatcg acagaacagt ccttaatcca |
| 1681 | gaaacctgaa atgaaggaag aggagactct gcgcagagca ctttgggtcc ggagggcgag |
| 1741 | actccggcgg aagcattccc gggcgggtga cccagcacgg tccctcttgg aattggattc |
| 1801 | gccattttat ttttcttgct gctaaatcac cgagcccgga agattagaga gttttatttc |
| 1861 | tgggattcct gtagacacac ccacccacat acatacattt atatatatat atattatata |
| 1921 | tatataaaaa taaatatctc tattttatat atataaaata tatatattct ttttttaaat |
| 1981 | taacagtgct aatgttattg gtgtcttcac tggatgtatt tgactgctgt ggacttgagt |
| 2041 | tgggagggga atgttcccac tcagatcctg acagggaaga ggaggagatg agagactctg |
| 2101 | gcatgatctt ttttttgtcc cacttggtgg ggccagggtc ctctcccctg cccaggaatg |
| 2161 | tgcaaggcca gggcatgggg gcaaatatga cccagttttg ggaacaccga caaacccagc |
| 2221 | cctggcgctg agcctctcta ccccaggtca gacggacaga aagacagatc acaggtacag |
| 2281 | ggatgaggac accggctctg accaggagtt tggggagctt caggacattg ctgtgctttg |
| 2341 | gggattccct ccacatgctg cacgcgcatc tcgcccccag gggcactgcc tggaagattc |
| 2401 | aggagcctgg gcggccttcg cttactctca cctgcttctg agttgcccag gagaccactg |
| 2461 | gcagatgtcc cggcgaagag aagagacaca ttgttggaag aagcagccca tgacagctcc |
| 2521 | ccttcctggg actcgccctc atcctcttcc tgctcccctt cctggggtgc agcctaaaag |
| 2581 | gacctatgtc ctcacaccat tgaaaccact agttctgtcc ccccaggaga cctggttgtg |
| 2641 | tgtgtgtgag tggttgacct tcctccatcc cctggtcctt cccttccctt cccgaggcac |
| 2701 | agagagacag ggcaggatcc acgtgcccat tgtggaggca gagaaaagag aaagtgtttt |
| 2761 | atatacggta cttatttaat atcccttttt aattagaaat taaaacagtt aatttaatta |
| 2821 | aagagtaggg ttttttttca gtattcttgg ttaatattta atttcaacta tttatgagat |
| 2881 | gtatcttttg ctctctcttg ctctcttatt tgtaccggtt tttgtatata aaattcatgt |
| 2941 | ttccaatctc tctctccctg atcggtgaca gtcactagct tatcttgaac agatatttaa |
| 3001 | ttttgctaac actcagctct gccctccccg atcccctggc tccccagcac acattccttt |
| 3061 | gaaataaggt ttcaatatac atctacatac tatatatata tttggcaact tgtatttgtg |
| 3121 | tgtatatata tatatatatg tttatgtata tatgtgattc tgataaaata gacattgcta |
| 3181 | ttctgttttt tatatgtaaa aacaaaacaa gaaaaaatag agaattctac atactaaatc |
| 3241 | tctctccttt tttaatttta atatttgtta tcatttattt attggtgcta ctgtttatcc |
| 3301 | gtaataattg tggggaaaag atattaacat cacgtctttg tctctagtgc agtttttcga |
| 3361 | gatattccgt agtacatatt tatttttaaa caacgacaaa gaaatacaga tatatcttaa |
| 3421 | aaaaaaaaaa gcattttgta ttaaagaatt taattctgat ctcaaaaaaa aaaaaa |

Human Vascular Endothelial Growth Factor A (VEGFA), transcript variant 7, isoform g (VEGF₁₆₅b) is encoded by the following amino acid sequence (NCBI Accession No. NP_{—}001028928.1 and SEQ ID NO: 23):

MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQENPCGPCSERRKHLFVQDPQTCKCSCKNTDS
RCKARQLELNERTCRSLTRKD

Human Vascular Endothelial Growth Factor B (VEGFB), is encoded by the following mRNA sequence (NCBI Accession No. NM_{—}003377 and SEQ ID NO: 24):

| | |
|---|---|
| 1 | gcgatgcggg cgcccccggc gggcggcccc ggcgggcacc atgagccctc tgctccgccg |
| 61 | cctgctgctc gccgcactcc tgcagctggc ccccgcccag gcccctgtct cccagcctga |
| 121 | tgcccctggc caccagagga aagtggtgtc atggatagat gtgtatactc gcgctacctg |
| 181 | ccagccccgg gaggtggtgg tgcccttgac tgtggagctc atgggcaccg tggccaaaca |
| 241 | gctggtgccc agctgcgtga ctgtgcagcg ctgtggtggc tgctgccctg acgatggcct |
| 301 | ggagtgtgtg cccactgggc agcaccaagt ccggatgcag atcctcatga tccggtaccc |
| 361 | gagcagtcag ctgggggaga tgtccctgga agaacacagc cagtgtgaat gcagacctaa |
| 421 | aaaaaaggac agtgctgtga agccagacag ggctgccact ccccaccacc gtccccagcc |
| 481 | ccgttctgtt ccgggctggg actctgcccc cggagcaccc tccccagctg acatcaccca |
| 541 | tcccactcca gccccaggcc cctctgccca cgctgcaccc agcaccacca gcgccctgac |
| 601 | ccccggacct gccgctgccg ctgccgacgc cgcagcttcc tccgttgcca agggcggggc |
| 661 | ttagagctca acccagacac ctgcaggtgc cggaagctgc gaaggtgaca catggctttt |
| 721 | cagactcagc agggtgactt gcctcagagg ctatatccca gtgggggaac aaagaggagc |
| 781 | ctggtaaaaa acagccaagc ccccaagacc tcagcccagg cagaagctgc tctaggacct |
| 841 | gggcctctca gagggctctt ctgccatccc ttgtctccct gaggccatca tcaaacagga |
| 901 | cagagttgga agaggagact gggaggcagc aagaggggtc acataccagc tcaggggaga |
| 961 | atggagtact gtctcagttt ctaaccactc tgtgcaagta agcatcttac aactggctct |
| 1021 | tcctcccctc actaagaaga cccaaacctc tgcataatgg gatttgggct ttggtacaag |
| 1081 | aactgtgacc cccaaccctg ataaaagaga tggaaggaaa aaaaaaaaaa aaaaaaaaaa |
| 1141 | aaaaaaaaaa aaaaaaaaaa aaaaaaaaaa aa |

Human Vascular Endothelial Growth Factor B (VEGF-B), is encoded by the following amino acid sequence (NCBI Accession No. NP_{—}003368.1 and SEQ ID NO: 25):

MSPLLRRLLLAALLQLAPAQAPVSQPDAPGHQRKVVSWIDVYTRATCQPR
EVVVPLTVELMGTVAKQLVPSCVTVQRCGGCCPDDGLECVPTGQHQVRMQ
ILMIRYPSSQLGEMSLEEHSQCECRPKKKDSAVKPDRAATPHHRPQPRSV
PGWDSAPGAPSPADITHPTPAPGPSAHAAPSTTSALTPGPAAAAADAAAS
SVAKGGA

Human Vascular Endothelial Growth Factor C (VEGF-C), is encoded by the following mRNA sequence (NCBI Accession No. NM_{—}005429 and SEQ ID NO: 26):

| | |
|---|---|
| 1 | cggggaaggg gagggaggag ggggacgagg gctctggcgg gtttggaggg gctgaacatc |
| 61 | gcggggtgtt ctggtgtccc ccgccccgcc tctccaaaaa gctacaccga cgcggaccgc |
| 121 | ggcggcgtcc tccctcgccc tcgcttcacc tcgcgggctc cgaatgcggg gagctcggat |
| 181 | gtccggtttc ctgtgaggct tttacctgac acccgccgcc tttccccggc actggctggg |
| 241 | agggcgccct gcaaagttgg gaacgcggag ccccggaccc gctcccgccg cctccggctc |
| 301 | gcccaggggg ggtcgccggg aggagcccgg gggagaggga ccaggagggg cccgcggcct |
| 361 | cgcaggggcg cccgcgcccc cacccctgcc cccgccagcg gaccggtccc ccacccccgg |
| 421 | tccttccacc atgcacttgc tgggcttctt ctctgtggcg tgttctctgc tcgccgctgc |
| 481 | gctgctcccg ggtcctcgcg aggcgcccgc cgccgccgcc gccttcgagt ccggactcga |
| 541 | cctctcggac gcggagcccg acgcgggcga ggccacggct tatgcaagca aagatctgga |
| 601 | ggagcagtta cggtctgtgt ccagtgtaga tgaactcatg actgtactct acccagaata |
| 661 | ttggaaaatg tacaagtgtc agctaaggaa aggaggctgg caacataaca gagaacaggc |
| 721 | caacctcaac tcaaggacag aagagactat aaaatttgct gcagcacatt ataatacaga |
| 781 | gatcttgaaa agtattgata atgagtggag aaagactcaa tgcatgccac gggaggtgtg |
| 841 | tatagatgtg gggaaggagt ttggagtcgc gacaaacacc ttctttaaac ctccatgtgt |
| 901 | gtccgtctac agatgtgggg gttgctgcaa tagtgagggg ctgcagtgca tgaacaccag |
| 961 | cacgagctac ctcagcaaga cgttatttga aattacagtg cctctctctc aaggccccaa |
| 1021 | accagtaaca atcagttttg ccaatcacac ttcctgccga tgcatgtcta aactggatgt |
| 1081 | ttacagacaa gttcattcca ttattagacg ttccctgcca gcaacactac cacagtgtca |
| 1141 | ggcagcgaac aagacctgcc ccaccaatta catgtggaat aatcacatct gcagatgcct |
| 1201 | ggctcaggaa gattttatgt tttcctcgga tgctggagat gactcaacag atggattcca |
| 1261 | tgacatctgt ggaccaaaca aggagctgga tgaagagacc tgtcagtgtg tctgcagagc |
| 1321 | ggggcttcgg cctgccagct gtggacccca caaagaacta gacagaaact catgccagtg |
| 1381 | tgtctgtaaa aacaaactct tccccagcca atgtggggcc aaccgagaat ttgatgaaaa |
| 1441 | cacatgccag tgtgtatgta aaagaacctg ccccagaaat caacccctaa atcctggaaa |
| 1501 | atgtgcctgt gaatgtacag aaagtccaca gaaatgcttg ttaaaaggaa agaagttcca |
| 1561 | ccaccaaaca tgcagctgtt acagacggcc atgtacgaac cgccagaagg cttgtgagcc |
| 1621 | aggattttca tatagtgaag aagtgtgtcg ttgtgtccct tcatattgga aaagaccaca |
| 1681 | aatgagctaa gattgtactg ttttccagtt catcgatttt ctattatgga aaactgtgtt |
| 1741 | gccacagtag aactgtctgt gaacagagag acccttgtgg gtccatgcta acaaagacaa |
| 1801 | aagtctgtct ttcctgaacc atgtggataa ctttacagaa atggactgga gctcatctgc |
| 1861 | aaaaggcctc ttgtaaagac tggttttctg ccaatgacca aacagccaag attttcctct |
| 1921 | tgtgatttct ttaaaagaat gactatataa tttatttcca ctaaaaatat tgtttctgca |
| 1981 | ttcattttta tagcaacaac aattggtaaa actcactgtg atcaatattt ttatatcatg |
| 2041 | caaaatatgt ttaaaataaa atgaaaattg tattat |

Human Vascular Endothelial Growth Factor C (VEGF-C), is encoded by the following amino acid sequence (NCBI Accession No. NP_{—}005420.1 and SEQ ID NO: 27):

MHLLGFFSVACSLLAAALLPGPREAPAAAAAFESGLDLSDAEPDAGEATA
YASKDLEEQLRSVSSVDELMTVLYPEYWKMYKCQLRKGGWQHNREQANLN
SRTEETIKFAAAHYNTEILKSIDNEWRKTQCMPREVCIDVGKEFGVATNT
FFKPPCVSVYRCGGCCNSEGLQCMNTSTSYLSKTLFEITVPLSQGPKPVT
ISFANHTSCRCMSKLDVYRQVHSIIRRSLPATLPQCQAANKTCPTNYMWN
NHICRCLAQEDFMFSSDAGDDSTDGFHDICGPNKELDEETCQCVCRAGLR
PASCGPHKELDRNSCQCVCKNKLFPSQCGANREFDENTCQCVCKRTCPRN
QPLNPGKCACECTESPQKCLLKGKKFHHQTCSCYRRPCTNRQKACEPGFS
YSEEVCRCVPSYWKRPQMS

Human Vascular Endothelial Growth Factor D (VEGF-D), is encoded by the following mRNA sequence (NCBI Accession No. NM_{—}004469 and SEQ ID NO: 28):

| | |
|---|---|
| 1 | caagacttct ctgcattttc tgccaaaatc tgtgtcagat ttaagacaca tgcttctgca |
| 61 | agcttccatg aaggttgtgc aaaaaagttt caatccagag ttgggttcca gctttctgta |
| 121 | gctgtaagca ttggtggcca caccacctcc ttacaaagca actagaacct gcggcataca |
| 181 | ttggagagat ttttttaatt ttctggacat gaagtaaatt tagagtgctt tctaatttca |
| 241 | ggtagaagac atgtccacct tctgattatt tttggagaac attttgattt ttttcatctc |
| 301 | tctctcccca cccctaagat tgtgcaaaaa aagcgtacct tgcctaattg aaataatttc |
| 361 | attggatttt gatcagaact gattatttgg ttttctgtgt gaagttttga ggtttcaaac |
| 421 | tttccttctg gagaatgcct tttgaaacaa ttttctctag ctgcctgatg tcaactgctt |
| 481 | agtaatcagt ggatattgaa atattcaaaa tgtacagaga gtgggtagtg gtgaatgttt |
| 541 | tcatgatgtt gtacgtccag ctggtgcagg gctccagtaa tgaacatgga ccagtgaagc |
| 601 | gatcatctca gtccacattg gaacgatctg aacagcagat cagggctgct tctagtttgg |
| 661 | aggaactact tcgaattact cactctgagg actggaagct gtggagatgc aggctgaggc |
| 721 | tcaaaagttt taccagtatg gactctcgct cagcatccca tcggtccact aggtttgcgg |
| 781 | caactttcta tgacattgaa acactaaaag ttatagatga agaatggcaa agaactcagt |
| 841 | gcagccctag agaaacgtgc gtggaggtgg ccagtgagct ggggaagagt accaacacat |
| 901 | tcttcaagcc cccttgtgtg aacgtgttcc gatgtggtgg ctgttgcaat gaagagagcc |
| 961 | ttatctgtat gaacaccagc acctcgtaca tttccaaaca gctctttgag atatcagtgc |
| 1021 | ctttgacatc agtacctgaa ttagtgcctg ttaaagttgc caatcataca ggttgtaagt |
| 1081 | gcttgccaac agccccccgc catccatact caattatcag aagatccatc cagatccctg |
| 1141 | aagaagatcg ctgttcccat tccaagaaac tctgtcctat tgacatgcta tgggatagca |
| 1201 | acaaatgtaa atgtgttttg caggaggaaa atccacttgc tggaacagaa gaccactctc |
| 1261 | atctccagga accagctctc tgtgggccac acatgatgtt tgacgaagat cgttgcgagt |
| 1321 | gtgtctgtaa aacaccatgt cccaaagatc taatccagca ccccaaaaac tgcagttgct |
| 1381 | ttgagtgcaa agaaagtctg gagacctgct gccagaagca caagctattt cacccagaca |
| 1441 | cctgcagctg tgaggacaga tgcccctttc ataccagacc atgtgcaagt ggcaaaacag |
| 1501 | catgtgcaaa gcattgccgc tttccaaagg agaaaagggc tgcccagggg ccccacagcc |
| 1561 | gaaagaatcc ttgattcagc gttccaagtt ccccatccct gtcattttta acagcatgct |
| 1621 | gctttgccaa gttgctgtca ctgttttttt cccaggtgtt aaaaaaaaaa tccattttac |
| 1681 | acagcaccac agtgaatcca gaccaacctt ccattcacac cagctaagga gtccctggtt |
| 1741 | cattgatgga tgtcttctag ctgcagatgc ctctgcgcac caaggaatgg agaggagggg |
| 1801 | acccatgtaa tccttttgtt tagttttgtt tttgtttttt ggtgaatgag aaaggtgtgc |
| 1861 | tggtcatgga atggcaggtg tcatatgact gattactcag agcagatgag gaaaactgta |
| 1921 | gtctctgagt cctttgctaa tcgcaactct tgtgaattat tctgattctt ttttatgcag |
| 1981 | aatttgattc gtatgatcag tactgacttt ctgattactg tccagcttat agtcttccag |
| 2041 | tttaatgaac taccatctga tgtttcatat ttaagtgtat ttaaagaaaa taaacaccat |
| 2101 | tattcaagcc aaaaaaaaaa aaaaaaaa |

Human Vascular Endothelial Growth Factor D (VEGF-D), is encoded by the following amino acid sequence (NCBI Accession No. NP_{—}004460.1 and SEQ ID NO: 29):

MYREWVVVNVFMMLYVQLVQGSSNEHGPVKRSSQSTLERSEQQIRAASSL
EELLRITHSEDWKLWRCRLRLKSFTSMDSRSASHRSTRFAATFYDIETLK
VIDEEWQRTQCSPRETCVEVASELGKSTNTFFKPPCVNVFRCGGCCNEES
LICMNTSTSYISKQLFEISVPLTSVPELVPVKVANHTGCKCLPTAPRHPY
SIIRRSIQIPEEDRCSHSKKLCPIDMLWDSNKCKCVLQEENPLAGTEDHS
HLQEPALCGPHMMFDEDRCECVCKTPCPKDLIQHPKNCSCFECKESLETC
CQKHKLFHPDTCSCEDRCPFHTRPCASGKTACAKHCRFPKEKRAAQGPHS
RKNP

Human Placenta Growth Factor (PGF), is encoded by the following mRNA sequence (NCBI Accession No. NM_{—}002632 and SEQ ID NO: 30):

| | |
|---|---|
| 1 | ctgctgtctg cggaggaaac tgcatcgacg gacggccgcc cagctacggg aggacctgga |
| 61 | gtggcactgg gcgcccgacg gaccatcccc gggacccgcc tgcccctcgg cgccccgccc |
| 121 | cgccgggccg ctccccgtcg ggttccccag ccacagcctt acctacgggc tcctgactcc |
| 181 | gcaaggcttc cagaagatgc tcgaaccacc ggccggggcc tcggggcagc agtgagggag |
| 241 | gcgtccagcc ccccactcag ctcttctcct cctgtgccag gggctccccg ggggatgagc |
| 301 | atggtggttt tccctcggag ccccctggct cgggacgtct gagaagatgc cggtcatgag |
| 361 | gctgttccct tgcttcctgc agctcctggc cgggctggcg ctgcctgctg tgccccccca |
| 421 | gcagtgggcc ttgtctgctg ggaacggctc gtcagaggtg gaagtggtac ccttccagga |
| 481 | agtgtggggc cgcagctact gccgggcgct ggagaggctg gtggacgtcg tgtccgagta |
| 541 | ccccagcgag gtggagcaca tgttcagccc atcctgtgtc tccctgctgc gctgcaccgg |
| 601 | ctgctgcggc gatgagaatc tgcactgtgt gccggtggag acggccaatg tcaccatgca |
| 661 | gctcctaaag atccgttctg gggaccggcc ctcctacgtg gagctgacgt tctctcagca |
| 721 | cgttcgctgc gaatgccggc ctctgcggga gaagatgaag ccggaaagga ggagacccaa |
| 781 | gggcaggggg aagaggagga gagagaagca gagacccaca gactgccacc tgtgcggcga |
| 841 | tgctgttccc cggaggtaac ccaccccttg gaggagagag accccgcacc cggctcgtgt |
| 901 | atttattacc gtcacactct tcagtgactc ctgctggtac ctgccctcta tttattagcc |
| 961 | aactgtttcc ctgctgaatg cctcgctccc ttcaagacga ggggcaggga aggacaggac |
| 1021 | cctcaggaat tcagtgcctt caacaacgtg agagaaagag agaagccagc cacagacccc |
| 1081 | tgggagcttc cgctttgaaa gaagcaagac acgtggcctc gtgaggggca agctaggccc |
| 1141 | cagaggccct ggaggtctcc aggggcctgc agaaggaaag aagggggccc tgctacctgt |
| 1201 | tcttgggcct caggctctgc acagacaagc agcccttgct ttcggagctc ctgtccaaag |
| 1261 | tagggatgcg gatcctgctg gggccgccac ggcctggctg gtgggaaggc cggcagcggg |
| 1321 | cggaggggat ccagccactt ccccctcttc ttctgaagat cagaacattc agctctggag |
| 1381 | aacagtggtt gcctgggggc ttttgccact ccttgtcccc cgtgatctcc cctcacactt |
| 1441 | tgccatttgc ttgtactggg acattgttct ttccggccaa ggtgccacca ccctgCCCCC |
| 1501 | cctaagagac acatacagag tgggccccgg gctggagaaa gagctgcctg gatgagaaac |
| 1561 | agctcagcca gtggggatga ggtcaccagg ggaggagcct gtgcgtccca gctgaaggca |
| 1621 | gtggcagggg agcaggttcc ccaagggccc tggcaccccc acaagctgtc cctgcagggc |
| 1681 | catctgactg ccaagccaga ttctcttgaa taaagtattc tagtgtggaa aaaaaaaaaa |
| 1741 | aaaaaaaaaa aaaaaaaa |

Human Placenta Growth Factor (PGF), is encoded by the following amino acid sequence (NCBI Accession No. NP_{—}002623.2 and SEQ ID NO: 31):

MPVMRLFPCFLQLLAGLALPAVPPQQWALSAGNGSSEVEVVPFQEVWGRS
YCRALERLVDVVSEYPSEVEHMFSPSCVSLLRCTGCCGDENLHCVPVETA
NVTMQLLKIRSGDRPSYVELTFSQHVRCECRPLREKMKPERRRPKGRGKR
RREKQRPTDCHLCGDAVPRR

### Therapeutic Methods

The methods of the invention are used to treat inflammatory disorders. As used herein the term “inflammatory disorders” is defined as any condition in which at least one tissue or system within a subject experienced inflammation. Furthermore, the term “inflammation” is defined for the purposes of the invention as any intrusion of an immune cell into a target tissue which is not part of the immune system. For instance, acute inflammation, or short-term inflammation, is characterized by infiltration of tissues by plasma and leukocytes. The process of acute inflammation is initiated by the blood vessels local to the injured tissue, which alter to allow the exudation of plasma proteins and leukocytes into the surrounding tissue. Alternatively, or in addition, chronic inflammation, or long-term inflammation, is characterized by the infiltration of mononuclear immune cells (monocytes, macrophages, lymphocytes, and plasma cells), tissue destruction, and attempts at healing, which include angiogenesis and fibrosis. Both acute and chronic inflammation are encompassed by the term inflammation unless specified otherwise. Another sign or symptom of inflammation is vascular remodeling or angiogenesis. Nonlimiting examples of vascular changes that indicate inflammation and/or angiogenesis are increases in blood vessel number, size, surface area, and vascular leak (also considered hemorrhage). Exemplary inflammatory disorders of the invention include, but are not limited to, asthma, chronic obstructive pulmonary disease, adult respiratory distress syndrome, interstitial lung disease, chronic bronchitis, eosinophilic bronchitis, eosinophilic pneumonia, pneumonia, atopic dermatitis, atopy, allergic rhinitis, idiopathic pulmonary fibrosis, scleroderma, autoimmune diseases, chronic prostatitis, glomerulonephritis, hypersensitivities, inflammatory bowel diseases, pelvic inflammatory disease, reperfusion injury, rheumatoid arthritis, transplant rejection, vasculitis, allergies, myopathies, and cancer.

The methods of the invention are used to treat angiogenesis. As used herein the term “angiogenesis” is defined as the growth or remodeling of new blood vessels from pre-existing vessels. For the purposes of the invention, the terms and concepts of vasculogenesis (spontaneous blood-vessel formation of vascular structures from circulating or tissue-resident endothelial stem cells, angioblasts, which proliferate in to de novo endothelial cells), intussusception (new blood vessel formation by splitting off existing ones, also known a splitting angiogenesis), sprouting angiogenesis, and arteriogenesis (formation of medium-sized blood vessels possessing tunica media plus adventitia) are considered equivalents of angiogenesis (formation of thin-walled endothelium-lined structures with or without a muscular smooth muscle wall and pericytes/fibrocytes). Angiogenesis can be a normal and healthy function, however, compositions and methods of the invention are used to treat angiogenesis that either causes or contributes to the severity of a pathologic condition. Exemplary angiogenic disorders of the invention include, but are not limited to, cancer, wet age-relate macular degeneration (AMD), inflammation, prolonged or abortive wound healing, hemorrhage, diabetic blindness (retinopathy), rheumatoid arthritis, psoriasis, obesity, hemangiomas, endometriosis, and any condition in which the inappropriate, uncontrolled, or undesired growth or remodeling of blood vessels occurs.

The methods of the invention are used to treat VEGF-induced disorders. As used herein the term “VEGF-induced disorders” is defined as any condition in which the overexpression or over production of VEGF in at least one tissue or system within a subject causes a pathological condition either locally or systemically. VEGF-induced disorders are caused, for example, by genetic variations, mutations or disorders; medical conditions (e.g. cancer, asthma); therapeutic intervention (prescription drugs, treatment for heart attack); lifestyle choices (e.g. diet, exercise); age; and exposure to environmental agents (e.g. mutagens or carcinogens). Nonlimiting exemplary VEGF-induced disorders include Castleman's Disease, von Hippel-Lindau (VHL) disease, POEMS (polyneuropathy, organomegaly, endocrinopathy, monoclonal gammopathy, and skin changes), angiogenesis, inflammation, prolonged or abortive wound healing, hemorrhage, and wet AMD.

The methods of the invention are used to treat fibrotic disorders. As used herein the term “fibrotic disorders” is defined as any condition in which unwanted, abnormal, or inappropriate fibrosis occurs in at least one tissue or system of a subject. Furthermore, the term “fibrosis” is defined for the purposes of the invention as the abnormal formation of excess fibrous connective tissue in an organ or tissue. Signs and symptoms of fibrosis include, but are not limited to, fibroproliferative matrix molecule deposition, enhanced collagen accumulation, apoptosis, and any combination thereof. Nonlimiting examples of fibrotic disease are injection fibrosis (consequence of intramuscular injections), endomyocardial fibrosis, mediastinal fibrosis, myelofibrosis, retroperiotoneal fibrosis, progressive massive fibrosis (complication from coal worker's pneumoconiosis), nephrogenic systemic fibrosis, interstitial lung disease (ILD), idiopathic pulmonary fibrosis (IPF), scleroderma, radiation-induced pulmonary fibrosis, bleomycin lung, sarcoidosis, silicosis, pulmonary fibrosis (also familial pulmonary fibrosis), autoimmune disease, and graft transplant fibrosis (e.g. renal, heart, liver). Fibrosis is associated with multiple diseases and disorders. Fibrotic disorders of the invention encompass all conditions in which the fibrosis occurs as a primary or secondary disorder.

The methods of the invention are used to treat macular degeneration, preferably, wet age-related macular degeneration (abbreviated AMD or ARMD). As used herein the term “macular degeneration” is defined as any condition in which results in loss of vision in the center of the visual field as a result of damage to the retina of a subject. Furthermore, the term “damage” is defined for the purposes of the invention as a compression, blockade, infarction, necrosis, ischemia, or detachment of the retina. Wet AMD results from ingrowths of blood vessels from the choroids behind the retina, which can result in a detachment of the retina. Signs and symptoms of wet AMD include, but are not limited to, loss of vision within the center of the visual field corresponding to the center, or macula, of the retina. Furthermore, signs and symptoms of wet AMD include blood and protein leakage below the macula. Moreover, subjects with wet AMD experience blurred vision, vision loss (which can be rapid), central scotomas (shadows or missing areas of vision), metamorphopsia (distorted vision), difficulty discerning colors (for instance, dark versus light colors), and slow recovery of visual function after exposure to bright light. Bleeding, leaking, and scarring from the ingrowth of blood vessels eventually cause irreversible damage to photoreceptors and rapid loss of vision. Compositions of the invention are used to decrease the ingrowth of blood vessels into the retina from the choriocappillaries, and the corresponding leaking and scarring that this ingrowth causes. As such, compositions of the invention decrease, prevent, or reverse, a sign or symptom of wet AMD. Wet AMD is also called neovascular or exudative AMD.

The methods of the invention are used to enhance wound healing. As used herein the term “wound healing” is defined as the process of regenerating dermal or epidermal tissue in a subject. Furthermore, the term “regenerating” is defined for the purposes of the invention as restoring the tissue to a state in which it is capable of performing the either the function that the tissue performed prior to being damaged or the function(s) performed by the surrounding tissue. In one aspect of the invention, the process of wound healing is divided into separate phases that overlap in time including inflammatory, proliferative, and remodeling phases. The inflammatory phase involves the clearing of infectious agents and debris. The proliferative phase involves angiogenesis, collagen deposition, granulation tissue formation (which includes fibroplasia, the formation of a new extracellular matrix), epithelialization (coverage of the wound by migrating epithelial cells), and wound contraction. During the remodeling phase, collagen is remodeled and realigned along tension lines.

VEGF expression increases at the time of wound healing and induces angiogenesis. However, uncontrolled VEGF secretion leads to the formation of abnormal and undesired hyperpermeable capillary structures. Moreover, VEGF overproduction at the time of wound healing leads to inflammation at the wound site. The effect of VEGF induction in wound healing is a prolonged or abortive wound healing process. Compositions and methods of the invention are used to reduce the negative effects of VEGF overproduction in wound healing, and as such, enhance the wound healing process. MiRNA and miRNA inhibitor compositions of the invention are administered to decrease angiogenesis and inflammation that lead to hyperpermeable or leaky capillary structures and prolonged wound healing.

The methods of the invention are used to treat cancer. As used herein the term “cancer” is defined as any condition in which a subset of cells within at least one tissue proliferate at an inappropriately fast rate thereby forming an in situ, benign or malignant tumor. Cancers of the invention are solid or liquid. Moreover, cancers are isolated or metastatic. Cancers of the invention are described according to “stage,” for example, according to the TNM system (accepted by the International Union Against Cancer (UICC) and the American Joint Committee on Cancer (AJCC)) or by other art-recognized methods. Cancer stage refers to the severity of the cancer, based on factors such as the location of the primary tumor, tumor size, number of tumors, and lymph node involvement (spread of cancer into lymph nodes). Alternatively, or in addition, cancers of the invention are described according to tumor grade by art-recognized methods (see, National Cancer Institute, www.cancer.gov). Tumor grade is a system used to classify cancer cells in terms of how abnormal a tumor looks under a microscope and how quickly a tumor is likely to grow and spread. Many factors are considered when determining tumor grade, including the structure and growth pattern of the cells. The specific factors used to determine tumor grade vary with each type of cancer. Cancers are also described using histologic grade, also called differentiation, which refers to how much the tumor cells resemble normal cells of the same tissue type (see, National Cancer Institute, www.cancer.gov). Finally, cancers are described by nuclear grade, which refers to the size and shape of the nucleus in tumor cells and the percentage of tumor cells that are dividing (see, National Cancer Institute, www.cancer.gov). Each of these methods of determining the severity of a cancer also constitutes a compilation of signs or symptoms of the cancer.

Cancers of the invention are further described according to the degree to which a tumor has secreted growth factors, degraded the extracellular matrix, become vascularized, lost adhesion to juxtaposed tissues, or metastasized. For example, a cancer that has spread from one primary location to multiple secondary locations is a more life-threatening condition and the metastatic, or spreading, process increased the severity of the disorder. Alternatively, or in addition, the severity of a disorder such as cancer can be further increased when considering the difficulty of treating tumors of varying types and locations, e.g., inoperable tumors, those cancers which have greater access to multiple body systems (hematological and immunological tumors), and those which are the most resistant to traditional treatments are considered most severe.

Exemplary cancers include, but are not limited to, acute lymphoblastic leukemia, acute myeloid leukemia, adrenocortical carcinoma, adrenocortical carcinoma, AIDS-related cancers, AIDS-related lymphoma, anal cancer, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, mycosis fungoides, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, islet cell tumors (endocrine pancreas), Kaposi Sarcoma, kidney (renal cell) cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine sarcoma, skin cancer (nonmelanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilms Tumor.

As used herein, the term “treat” is meant to describe a process by which a sign or symptom of a disorder is eliminated. Alternatively, or in addition, a disorder which can occur in multiple locations, is treated if that disorder is eliminated within at least one of multiple locations.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder, which can occur in multiple locations, is alleviated if the severity of the disorder is decreased within at least one of multiple locations.

As used herein, the term “severity” is meant to describe an unfavorable prognosis for a subject, a progression of a disorder to a more deleterious stage, a presentation of a sign or symptom or a diagnosis of an additional or secondary disorder, a requirement for invasive, experimental, or high-risk medical treatment, an indication that the disorder has become systemic rather than local or that the disorder has invaded additional or secondary bodily systems, the potential of a disorder to transform from a benign to malignant state, or the potential of a disorder to escalate from a state that is managed by preventative, daily, or routine medicine to a crises state that is managed by emergency medicine or specialize care centers.

As used herein, the term “severity” is meant to describe the potential of cancer to transform from a precancerous, or benign, state into a malignant state. Alternatively, or in addition, severity is meant to describe, for instance, a cancer stage or grade. In additional aspects of the invention, severity describes the number and location of secondary cancers as well as the operability or drug-accessibility of those tumors. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

The pharmaceutically effective dose depends on the type of disease, the composition used, the route of administration, the individual and physical characteristics of the subject under consideration (for example, age, gender, weight, diet, smoking-habit, exercise-routine, genetic background, medical history, hydration, blood chemistry), concurrent medication, and other factors that those skilled in the medical arts will recognize.

Generally, an amount from about 0.01 mg/kg and 25 mg/kg body weight/day of active ingredients is administered dependent upon potency of the miRNA and/or the miRNA inhibitor, e.g. the therapeutic composition. In alternative embodiments dosage ranges include, but are not limited to, 0.01-0.1 mg/kg, 0.01-1 mg/kg, 0.01-10 mg/kg, 0.01-20 mg/kg, 0.01-30 mg/kg, 0.01-40 mg/kg, 0.01-50 mg/kg, 0.01-60 mg/kg, 0.01-70 mg/kg, 0.01-80 mg/kg, 0.01-90 mg/kg, 0.01-100 mg/kg, 0.01-150 mg/kg, 0.01-200 mg/kg, 0.01-250 mg/kg, 0.01-300 mg/kg, 0.01-500 mg/kg, and all ranges and points in between. In alternative embodiments dosage ranges include, but are not limited to, 0.01-1 mg/kg, 1-10 mg/kg, 10-20 mg/kg, 20-30 mg/kg, 30-40 mg/kg, 40-50 mg/kg, 50-60 mg/kg, 60-70 mg/kg, 70-80 mg/kg, 80-90 mg/kg, 90-100 mg/kg, 100-150 mg/kg, 150-200 mg/kg, 200-300 mg/kg, 300-500 mg/kg, and all ranges and points in between.

As used herein the term “symptom” is defined as an indication of disease, illness, or injury in the body. Symptoms are felt or noticed by the individual experiencing the symptom, but may not easily be noticed by others. Others are defined as non-health-care professionals.

As used herein the term “sign” is also defined as an indication of disease, illness, or injury in the body. Signs are defined as things that can be seen by a doctor, nurse, or other health care professional.

### Pharmaceutical Compositions

The invention provides a composition including at least one miRNA and/or a miRNA inhibitor and a pharmaceutically acceptable carrier. Pharmaceutically acceptable carriers are covalently or non-covalently bound, admixed, encapsulated, conjugated, operably-linked, or otherwise associated with the miRNA and/or miRNA inhibitor such that the pharmaceutically acceptable carrier increases the cellular uptake, stability, solubility, half-life, binding efficacy, specificity, targeting, distribution, absorption, or renal clearance of the miRNA and/or miRNA inhibitor. Alternatively, or in addition, the pharmaceutically acceptable carrier increases or decreases the immunogenicity of the miRNA and/or miRNA inhibitor. Furthermore, the pharmaceutically acceptable carrier is capable to increasing the cytotoxicity of the miRNA and/or miRNA inhibitor composition with respect to the targeted cancer cells.

Alternatively, or in addition, pharmaceutically acceptable carriers are salts (for example, acid addition salts, e.g., salts of hydrochloric, hydrobromic, acetic acid, and benzene sulfonic acid), esters, salts of such esters, or any other compound which, upon administration to a subject, are capable of providing (directly or indirectly) the biologically active compositions of the invention. As such, the invention encompasses prodrugs, and other bioequivalents. As used herein, the term “prodrug” is meant to describe, a pharmacological substance that is administered in an inactive (or significantly less active) form. Once administered, the prodrug is metabolised in vivo into an active metabolite. Pharmaceutically acceptable carriers are alternatively or additionally diluents, excipients, adjuvants, emulsifiers, buffers, stabilizers, and/or preservatives.

Pharmaceutically acceptable carriers of the invention are miRNA and/or miRNA inhibitor delivery systems/mechanisms that increase uptake of the miRNA and/or miRNA inhibitor by targeted cells. For example, pharmaceutically acceptable carriers of the invention are viruses, recombinant viruses, engineered viruses, viral particles, replication-deficient viruses, liposomes, cationic lipids, anionic lipids, cationic polymers, polymers, hydrogels, micro- or nano-capsules (biodegradable), micropheres (optionally bioadhesive), cyclodextrins, plasmids, mammalian expression vectors, proteinaceous vectors, or any combination of the preceding elements (see, O'Hare and Normand, International PCT Publication No. WO 00/53722; U.S. Patent Publication 2008/0076701). Moreover, pharmaceutically acceptable carriers that increase cellular uptake can be modified with cell-specific proteins or other elements such as receptors, ligands, antibodies to specifically target cellular uptake to a chosen cell type.

In another aspect of the invention, compositions are first introduced into a cell or cell population that is subsequently administered to a subject. In some embodiments, a miRNA and/or miRNA inhibitor is delivered intracellularly, e.g., in cells of a target tissue such as lung, or in inflamed tissues. Included within the invention are compositions and methods for delivery of an isolated miRNA and/or miRNA inhibitor and/or composition by removing cells of a subject, delivering the isolated miRNA and/or miRNA inhibitor or composition to the removed cells, and reintroducing the cells into a subject. In some embodiments, a miRNA and/or miRNA inhibitor molecule is combined with a cationic lipid or transfection material such as LIPOFECTAMINE (Invitrogen).

In one aspect, the active compounds are prepared with pharmaceutically acceptable carriers that will protect the miRNA and/or miRNA inhibitor molecule against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Pharmaceutically acceptable carriers are cationic lipids that are bound or associated with miRNA and/or miRNA inhibitor. Alternatively, or in addition, miRNAs and/or miRNA inhibitors are encapsulated or surrounded in cationic lipids, e.g. lipsosomes, for in vivo delivery. Exemplary cationic lipids include, but are not limited to, N41-(2,3-dioleoyloxy)propyliN,N,N-trimethylammonium chloride (DOTMA); 1,2-bis(oleoyloxy)-3-3-(trimethylammonium)propane (DOTAP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N-(N′,N′-dimethylaminoethane)carbamoyl)cholesterol (DC-Chol); 3.beta.-[N′,N′-diguanidinoethyl-aminoethane)carbamoyl cholesterol (BGTC); 2-(2-(3-(bis(3-aminopropyl)amino)propylamino)acetamido)-N,N-ditetradecyla-cetamide (RPR209120); pharmaceutically acceptable salts thereof, and mixtures thereof. Further exemplary cationic lipids include, but are not limited to, 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Exemplary polycationic lipids include, but are not limited to, tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof. Further examplary polycationic lipids include, but are not limited to, 2,5-bis(3-aminopropylamino)-N-(2-(dioctadecylamino)-2-oxoethyl)pentanamid-e (DOGS); 2,5-bis(3-aminopropylamino)-N-(2-(di(Z)-octadeca-9-dienylamino)-2-oxoethyl)pentanamide (DOGS-9-en); 2,5-bis(3-aminopropylamino)-N-(2-(di(9Z,12Z)-octadeca-9,12-dienylamino)-2-oxoethyl)pentanamide (DLinGS); 3-beta-(N.sup.4-(N.sup.1, N.sup.8-dicarbobenzoxyspermidine)carbamoyl)chole-sterol (GL-67); (9Z,9^{y}Z)-2-(2,5-bis(3-aminopropylamino)pentanamido)propane-1,3-diyl-dioct-adec-9-enoate (DOSPER); 2,3-dioleyloxy-N-[2(sperminecarboxamido)ethyl]-N,N-dimethyl-1-propanamini-urn trifluoro-acetate (DOSPA); pharmaceutically acceptable salts thereof, and mixtures thereof.

Examples of cationic lipids are described in U.S. Pat. Nos. 4,897,355; 5,279,833; 6,733,777; 6,376,248; 5,736,392; 5,334,761; 5,459,127; 2005/0064595; U.S. Pat. Nos. 5,208,036; 5,264,618; 5,279,833; 5,283,185; 5,753,613; and 5,785,992; each of which is incorporated herein in its entirety.

Pharmaceutically acceptable carriers of the invention also include non-cationic lipids, such as neutral, zwitterionic, and anionic lipids. Examplary non-cationic lipids include, but are not limited to, 1,2-Dilauroyl-sn-glycerol (DLG); 1,2-Dimyristoyl-snglycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-snglycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-0-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoylsn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-snglycero-3-phospho-sn-1-glycerol (sodium salt; DSP-sn-1-G); 1,2-Dipalmitoyl-snglycero-3-phospho-L-serine (sodium salt; DPP S); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-snglycero-3-phosphoglycerol (ammonium salt; POPG); 1-Palmitoyl-2-4-o-sn-glycero-3-phosphocholine (P-lyso-PC); 1-Stearoyl-2-lyso-sn-glycero-3-phosphocholine (S-lysoPC); and mixtures thereof. Further examplary non-cationic lipids include, but are not limited to, polymeric compounds and polymer-lipid conjugates or polymeric lipids, such as pegylated lipids, including polyethyleneglycols, N-(Carbonylmethoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N(Carbonyl-methoxypolyethyleneglycol 2000)-1,2-distearoyl-sn-glycero-3-phosphoethanolamine (sodium salt; DSPE-MPEG-2000); N-(Carbonylmethoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof. Examples of non-cationic lipids include, but are not limited to, dioleoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Pharmaceutically-acceptable carriers of the invention further include anionic lipids. Examplary anionic lipids include, but are not limited to, phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

Supplemental or complementary methods for delivery of nucleic acid molecules for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any nucleic acid or inhibitor molecule of the invention.

Pharmaceutical compositions are administered locally and/or systemically. As used herein, the term “local administration” is meant to describe the administration of a pharmaceutical composition of the invention to a specific tissue or area of the body with minimal dissemination of the composition to surrounding tissues or areas. Locally administered pharmaceutical compositions are not detectable in the general blood stream when sampled at a site not immediate adjacent or subjacent to the site of administration.

As used herein the term “systemic administration” is meant to describe in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant disclosure can potentially localize the drug, e.g., in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

A pharmaceutically acceptable carrier is chosen to be compatible with its intended route of administration. Examples of routes of administration include parenteral, e.g., intravenous, intradermal, subcutaneous, oral (e.g., inhalation or insufflation), transdermal (topical), transmucosal, transopthalmic, tracheal, intranasal, epidermal, intraperitoneal, intraorbital, intraarterial, intracapsular, intraspinal, intrasternal, intracranial, intrathecal, intraventricular, and rectal administration. Alternatively, or in addition, compositions of the invention are administered non-parentally, for example, orally. Alternatively, or further in addition, compositions of the invention are administered surgically, for example, as implants or biocompatible polymers.

Pharmaceutical compositions are administered via injection or infusion, e.g. by use of an infusion pump. Direct injection of the nucleic acid molecules of the invention, is performed using standard needle and syringe methodologies, or by needle-free technologies such as those described in Conry et al., Clin. Cancer Res. 5:2330-2337, 1999 and Barry et al., International PCT Publication No. WO 99/31262.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

An isolated nucleic acid with a pharmaceutically acceptable carrier of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the subject should preferably be closely monitored during and for a reasonable period after treatment.

Compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof.

The pharmaceutical compositions are in the form of a sterile injectable aqueous or oleaginous suspension. This suspension is formulated according to the known art using those suitable dispersing or wetting agents and suspending agents that have been mentioned above. The sterile injectable preparation is a sterile injectable solution or suspension in a non-toxic parentally acceptable diluent or solvent, e.g., as a solution in 1,3-butanediol. Exemplary acceptable vehicles and solvents are water, Ringer's solution and isotonic sodium chloride solution. In addition, sterile, fixed oils are conventionally employed as a solvent or suspending medium. For this purpose, any bland fixed oil is employed including synthetic mono- or diglycerides. In addition, fatty acids such as oleic acid are used in the preparation of injectables.

Sterile injectable solutions can be prepared by incorporating the miRNA and/or miRNA inhibitor in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. MiRNA and/or miRNA inhibitors containing at least one 2′-O-methoxyethyl modification are used when formulating compositions for oral administration. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Exemplary penetrants for transdermal administration include, but are not limited to, lipids, liposomes, fatty acids, fatty acid, esters, steroids, chelating agents, and surfactants. Preferred lipids and liposomes of the invention are neutral, negative, or cationic. Compositions are encapsulated within liposomes or form complexes thereto, such as cationic liposomes.

Alternatively, or in addition, compositions are complexed to lipids, such as cationic lipids. Compositions prepared for transdermal administration are provided by iontophoresis. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into patches, ointments, lotions, salves, gels, drops, sprays, liquids, powders, or creams as generally known in the art.

Pharmaceutical compositions of the invention are administered systemically and are intended to cross the blood-brain barrier to contact cells of the central nervous system. Alternatively, or in addition, pharmaceutical compositions are administered intraspinally by, for example, lumbar puncture, or intracranially, e.g. intrathecally or intraventricularly. By the preceding routes, pharmaceutical compositions are introduced directly into the cerebral spinal fluid. Nonlimiting examples of agents suitable for formulation with the nucleic acid molecules of the invention, particularly for targeting nervous system tissues, include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly (DL-lactidecoglycolide) microspheres for sustained release delivery after intracerebral implantation (Emerich, D. F., et al., Cell Transplant 8:47-58, 1999) (Alkermes, Inc. Cambridge, Mass.); and loaded nanoparticles, such as those made of polybutylcyanoacrylate, which can deliver drugs across the blood brain barrier and can alter neuronal uptake mechanisms (Prog. Neuropsychopharmacol Biol. Psychiatry 23:941-949, 1999). Other non-limiting examples of delivery strategies for the nucleic acid molecules of the instant disclosure include material described in Boado, et al., J. Pharm. Sci. 87:1308-1315, 1998; Tyler, et al., FEBS Lett. 421:280-284, 1999; Pardridge, et al, PNAS USA. 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA. 96:7053-7058, 1999.

The miRNAs and/or miRNA inhibitors and compositions of the invention are also administered in the form of suppositories, e.g., for rectal administration of the drug. These compositions are prepared by mixing the drug with a suitable non-irritating excipient that is solid at ordinary temperatures but liquid at the rectal temperature and will therefore melt in the rectum to release the drug. Such materials include cocoa butter and polyethylene glycols.

Aqueous suspensions contain the active materials in admixture with excipients suitable for the manufacture of aqueous suspensions. Such excipients are suspending agents, e.g., sodium carboxymethylcellulose, methylcellulose, hydropropylmethylcellulose, sodium alginate, polyvinylpyrrolidone, gum tragacanth and gum acacia; dispersing or wetting agents can be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, e.g., polyethylene sorbitan monooleate. The aqueous suspensions also contain one or more preservatives, e.g., ethyl, or n-propyl p-hydroxybenzoate, one or more coloring agents, one or more flavoring agents, and one or more sweetening agents, such as sucrose or saccharin.

Oily suspensions are formulated by suspending the active ingredients in a vegetable oil, e.g., arachis oil, olive oil, sesame oil or coconut oil, or in a mineral oil such as liquid paraffin. The oily suspensions contain a thickening agent, e.g., beeswax, hard paraffin or cetyl alcohol. Sweetening agents and flavoring agents are added to provide palatable oral preparations. These compositions are preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, e.g., sweetening, flavoring and coloring agents, are also present.

Pharmaceutical compositions of the invention are in the form of oil-in-water emulsions. The oily phase is a vegetable oil or a mineral oil or mixtures of these. Suitable emulsifying agents are naturally-occurring gums, e.g., gum acacia or gum tragacanth, naturally-occurring phosphatides, e.g., soy bean, lecithin, and esters or partial esters derived from fatty acids and hexitol, anhydrides, e.g., sorbitan monooleate, and condensation products of the said partial esters with ethylene oxide, e.g., polyoxyethylene sorbitan monooleate. The emulsions also contain sweetening and flavoring agents.

In a preferred aspect, the pharmaceutically acceptable carrier can be a solubilizing carrier molecule. More preferably, the solubilizing carrier molecule can be Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof, beta-cyclodextrin or analogs thereof, and gamma-cyclodextrin or analogs thereof.

The invention also provides compositions prepared for storage or administration. Acceptable carriers or diluents for therapeutic use are well known in the pharmaceutical art, and are described, e.g., in Remington's Pharmaceutical Sciences, Mack Publishing Co., A. R. Gennaro Ed., 1985. For example, preservatives, stabilizers, dyes and flavoring agents are provided. These include sodium benzoate, sorbic acid and esters of phydroxybenzoic acid. In addition, antioxidants and suspending agents are used.

### EXAMPLES

### Example 1

### General Methods

### RNA Extraction:

Lungs: Transgenic mice were sacrificed and lungs were removed en block, minced, stored in Trizol® (Invitrogen) and flash-frozen in liquid nitrogen. Subsequently, stored tissues were thawed on ice, homogenized with a tissue homogenizer and the aqueous phase containing total RNA was separated after adding chloroform to the tissue homogenate according to the Trizol® kit instructions. Total RNA (containing microRNA was extracted from the aqueous phase with mirVana™ miRNA isolation kit (Ambion) according to the manufacturer's instructions.

### MicroRNA Array Hybridization:

Small size RNAs (<40 nt) were separated from total RNA by PAGE purification using Flash-PAGE™ fractionator (Ambion), tailed with poly-A and coupled to fluorescent dyes (Cy-3 and Cy-5, Amersham) using mirVana™ miRNA labeling kit (Ambion). Labeled microRNAs were hybridized to spotted mirVana™ microRNA arrays (Ambion) according to the manufacturer's instructions and hybridized arrays were scanned after 14 hours incubation in a 42° C. water bath.

### Quantitative RT-PCR:

RNAs extracted from lung tissue or cultured cells were subjected to reverse transcription with stem loop primers and subsequently quantitative PCR using corresponding Taqman® microRNA assays (Applied Biosystems), according to manufacturer's instructions. Expression levels are presented as relative levels calculated as the expression level of the gene in question compared to the expression of a normalizer gene (a stable small RNA sno202 for miRNA).

### Cell Culture:

MLECs (Mouse lung endothelial cells) were a gift from Dr P. Lee (Yale University). These are primary mouse lung endothelial cells that were immuno-isolated from lungs and cultured in DMEM-F12 (Invitrogen) with 20% fetal calf serum as described previously (Journal of Clinical Investigation, 116: 3050-3059, 2006).

MLE-12 cells are mouse lung epithelial cells and were purchased from the American Type Culture Collection (ATCC).

PASMC cells (rat primary pulmonary artery smooth muscle cells) have been described previously (Am J Physiol Lung Cell Mol Physiol, 283: L815-L829, 2002) and were a gift from Dr. P. Lee.

VEGF provided to cells for in vitro studies was recombinant human VEGF₁₆₅ from NCI, Lot No. 1130071, given at 100-150 ng/ml.

### MicroRNA Supplementation:

RNA molecules: MiR-1 RNA mimic (sense, UGGAAUGUAAAGAAGUAUGUAA, SEQ ID NO: 32); antisense, ACAUACUUCUUUACAUUCAAUA, (SEQ ID NO: 33) was synthesized by Dharmacon.

AllStars negative control siRNA (Qiagen, sense, GGGUAUCGACGAUUACAAAdTdT, SEQ ID NO: 34; antisense, UUUGUAAUCGUCGAUACCCdTdG, SEQ ID NO: 35) was purchased and used as negative control double stranded RNA molecule in mouse supplementation experiments.

Six week-old lung targeted VEGF₁₆₅ transgenic mice (Nat Med, 10(10): 1095-1103) and their transgene-negative littermate controls received intranasal inhalational treatment with double stranded miR-1 RNA mimic (2 mg/kg body weight) or siRNA buffer (5× buffer from Dharmacon, 300 mM KCL, 30 mM HEPES-pH 7.5, 1.0 mM MgCl₂) as described previously (Journal of Biological Chemistry, 279(11):10677-10684, 2004), every day for 10 days. Doxycycline (0.5 mg/ml) was added to their drinking water after the first treatment to induce the VEGF transgene. The mice were sacrificed on the day 10, lungs and trachea removed and BAL collected for further analysis as described previously (Nat Med, 10(10):1095-1103).

### MicroRNA Delivery:

MiRNA compositions of the invention are delivered by a variety of means. In a preferred embodiment of the invention, miRNA compositions are delivered by viral-mediated delivery. Compositions of the invention are contacted, incorporated into, or enclosed within viral particle (or virus-like particle) or replication-defective virus (engineered virus) prior to administration. Following administration of the viral particle or engineered virus, the miRNA composition is injected into at least one cell. Alternatively, or in addition, the miRNA composition is transported across the plasma membrane of at least one cell. Virus like particles (VLPs) consist of viral protein(s) derived from the structural proteins of a virus. In some cases these proteins are embedded within a lipid bilayer. These particles resemble the virus from which they were derived but lack viral nucleic acid and are not infectious. All known viruses are contemplated. Viral delivery is achieved using art-recognized methods.

### Example 2

### MicroRNA Analysis of VEGF₁₆₅ Transgene (−) and VEGF₁₆₅ Transgene (+) Mice

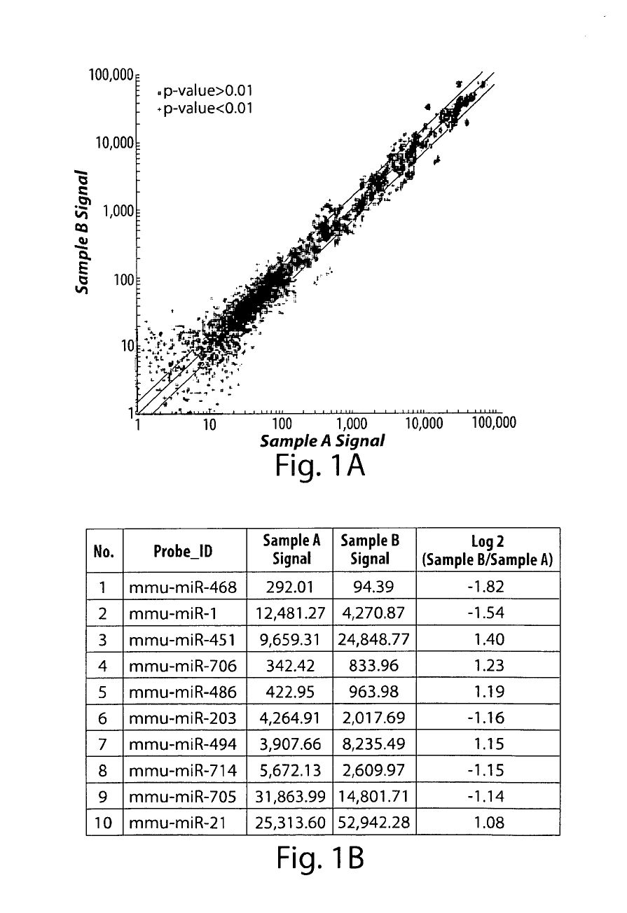

A microRNA (miRNA) microarray analysis comparing VEGF₁₆₅ transgene (−) (Sample A) and VEGF₁₆₅ transgene (+) (Sample B) mice was performed (FIG. 1). Total RNA was extracted from lung tissue of VEGF₁₆₅ transgene (−) and VEGF₁₆₅ transgene (+) mice. From these pools of total RNA, small size RNAs (<40 nt) were separated, tailed with poly-A and coupled to fluorescent dyes (Cy-3 and Cy-5). Fluorescently-labeled miRNAs were then hybridized to spotted mirVana™ microRNA arrays. The resulting hybridized arrays were scanned.

The intensity of the miRNA fluorescent signal, which is directly proportional to the abundance of that miRNA in the corresponding lung tissue, was calculated from Sample A (VEGF₁₆₅ transgene (−)) and Sample B (VEGF₁₆₅ transgene (+)). A ratio was calculated for each miRNA between the two conditions (log 2 (Sample B/Sample A)). As indicated by the graph in FIG. 1A and by the numerical scores for the individual signals from each sample as well as the calculated ratios presented in FIG. 1B, the abundances of several miRNAs are skewed in one sample over another, i.e. certain miRNAs are strongly up- or down-regulated in the transgenic mouse. MiR-1, for instance, is abundant in the VEGF₁₆₅ transgene (−) (Sample A) and significantly less abundant in the VEGF₁₆₅ transgene (+) (Sample B). MiR-203 demonstrates a similar pattern of expression to miR-1, however, the overall expression level is significantly less (approximately three-fold). MiR-21, for example, demonstrates an opposite pattern of expression to miR-1 and miR-203. MiR-21 is nearly twice as abundant in the VEGF₁₆₅ transgene (+) mouse than in the negative control.

In summary, approximately half of the miRNAs in Table 1B are more abundantly expressed in the negative control (miR-468, miR-1, miR-203, miR-714, miR-705) whereas the other half are more abundantly expressed in the VEGF₁₆₅ transgene (+) mouse lung (miR-451, miR-706, miR-486, miR-494, miR-21).

### Example 3

### MiRNA Expression Levels in VEGF₁₆₅ Transgene (+) Mouse Lung

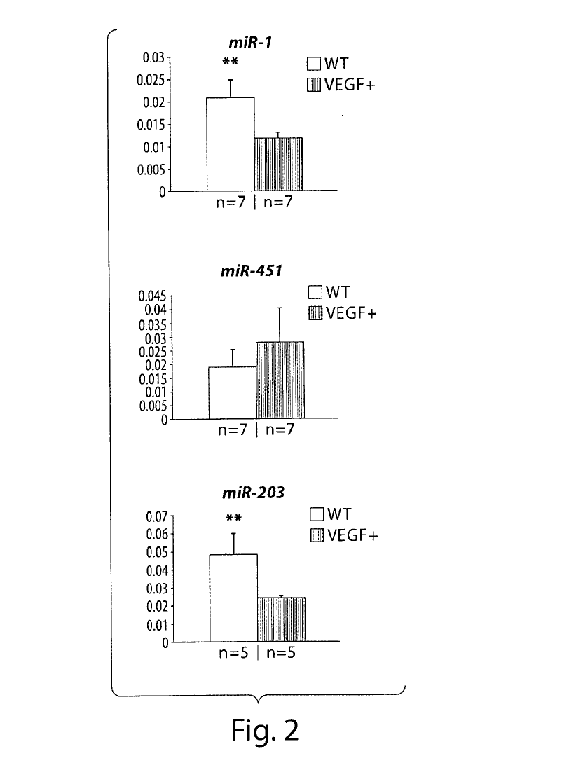

A series of real time quantitative polymerase chain reaction (qPCR) evaluations of the endogenous expression levels of miRNAs miR-1, miR-451 and miR-203 in the lung tissue of VEGF₁₆₅ transgene (−) and VEGF₁₆₅ transgene (+) mice was performed (FIG. 2). The data demonstrate a statistically significant decrease in the expression of miR-1 and miR-203 in VEGF₁₆₅ transgene (+) lung tissue compared to VEGF₁₆₅ transgene (−) lung control (left and right panels, respectively). The expression of miR-451 was not statistically different between these two genetic backgrounds in lung tissue (middle panel).

### Example 4

### MiRNA Expression Levels in Lung Endothelial Cells in the Presence and Absence of VEGF

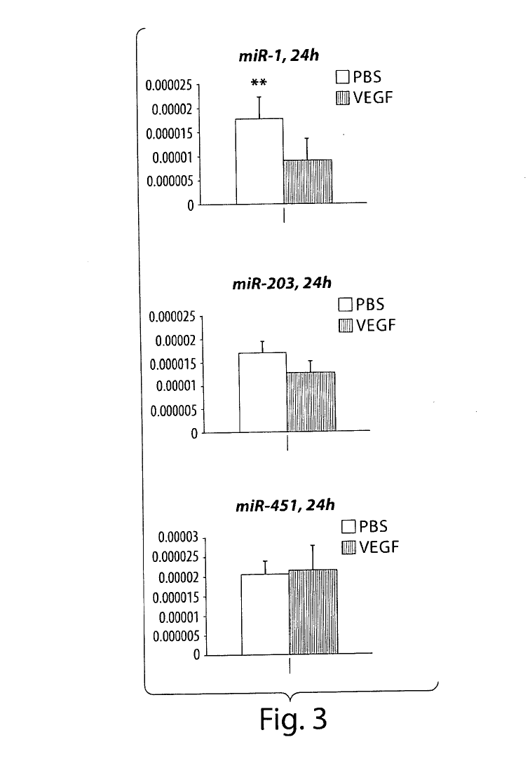

A series of real time quantitative polymerase chain reaction (qPCR) evaluations of the expression levels of miRNAs miR-1, miR-451 and miR-203 in lung endothelial cells was performed following incubations with either VEGF or negative control (PBS) (FIG. 3). Mouse lung endothelial cells (MLECs) are primary cells that were immuno-isolated from lungs. The data demonstrate that the expression of miR-1 decreases significantly following a 24-hour incubation with VEGF versus PBS negative control (left panel). This effect was not observed with miR-203 or miR-451 (middle and right panels, respectively).

### Example 5

### MiRNA Expression Levels in Pulmonary Artery Smooth Muscle Cells the Presence and Absence of VEGF

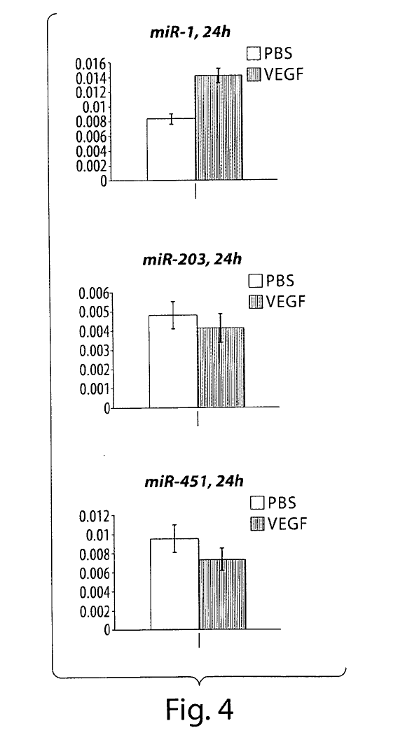

A series of real time quantitative polymerase chain reaction (qPCR) evaluations of the expression levels of miRNAs miR-1, miR-451 and miR-203 in pulmonary artery smooth muscle cells was performed following incubations with either VEGF or negative control (PBS) (FIG. 4). Rat primary pulmonary artery smooth muscle (PASMC) cells were used. The data demonstrate that the expression of miR-1 increases following a 24-hour incubation with VEGF compared to negative control (left panel).

### Example 6

### MiRNA Expression Levels in Pulmonary Artery Smooth Muscle Cells the Presence and Absence of VEGF

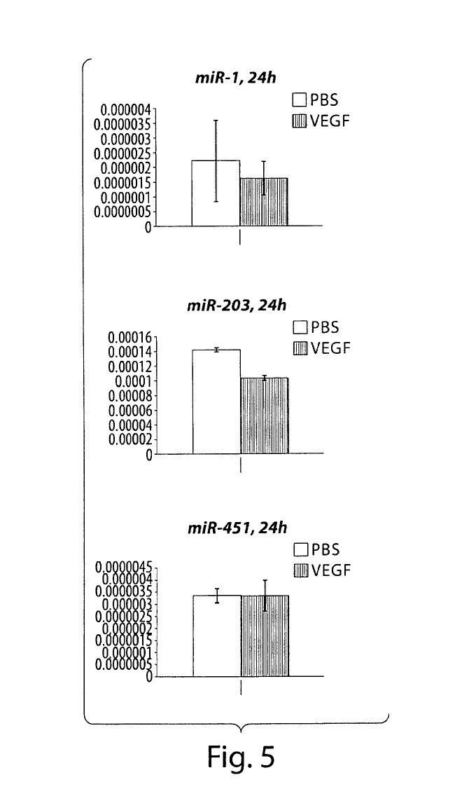

A series real time quantitative polymerase chain reaction (qPCR) evaluations of the expression levels of miRNAs miR-1, miR-451 and miR-203 in lung epithelial cells was performed following incubations with either VEGF or negative control (PBS) (FIG. 5). Mouse lung epithelial (MLE-12) cells were used. The data demonstrate that there is no statistically significant change in the level of miR-1 or miR-451 (left and middle panel) but miR-203 level decreases following incubation with VEGF compared to negative control (PBS) (right panel).

### Example 7

### MiR-1 Supplementation Decreases BAL Hemorrhage in VEGF₁₆₅ Transgenic Mice

Six week-old lung-targeted VEGF₁₆₅ transgenic mice (+) and their transgene-negative littermate controls, VEGF₁₆₅ transgenic mice (−), received intranasal inhalational treatment with a double stranded miR-1 RNA mimic (2 mg/kg body weight) or siRNA buffer molecule every day for 10 days. Doxycycline (0.5 mg/ml) was added to their drinking water after the first treatment to induce the VEGF transgene. Following sacrifice on the day 10, bronchoalveolar (BAL) fluid was collected for analysis. VEGF-induced angiogenesis in the airway is associated with large friable vessels that bleed easily. Bleeding can be seen as a red (dark) color in the bronchoalveolar fluid.

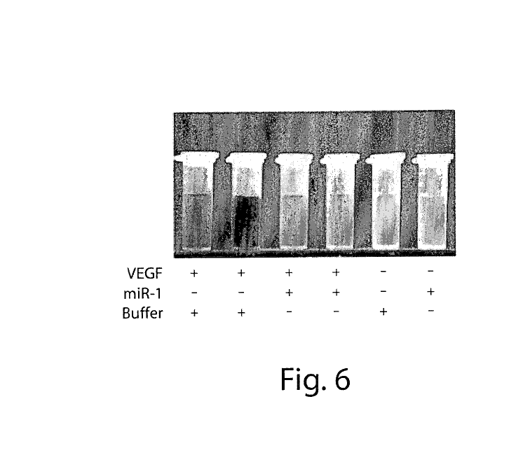

FIG. 6 shows that bronchoalveolar lavage (BAL) hemorrhage occurs in VEGF₁₆₅ transgene (+) mice (fluid from transgene+mice, left four vials), however, the amount of the BAL hemorrhage observed is significantly decreased by miR-1 supplementation (middle two vials). Supplementation with buffer alone in the absence of miR-1 is not sufficient to decrease or inhibit BAL hemorrhage (left two vials). BAL hemorrhage was not observed in the VEGF₁₆₅ transgene (−) mice (right two vials).

### Example 8

### MiR-1 Supplementation Decreases Abundance of Inflammatory Cell Types in the BAL Fluid of VEGF₁₆₅ Transgenic Mice

The abundances of cells within a number of inflammatory cell types (macrophage, lymphocyte, eosinophil, and neutrophil cells) were determined following collection of BAL fluid from either VEGF₁₆₅ transgene (+) or VEGF₁₆₅ transgene (−) mice supplemented with MiR-1 or siRNA buffer as described in Example 7.

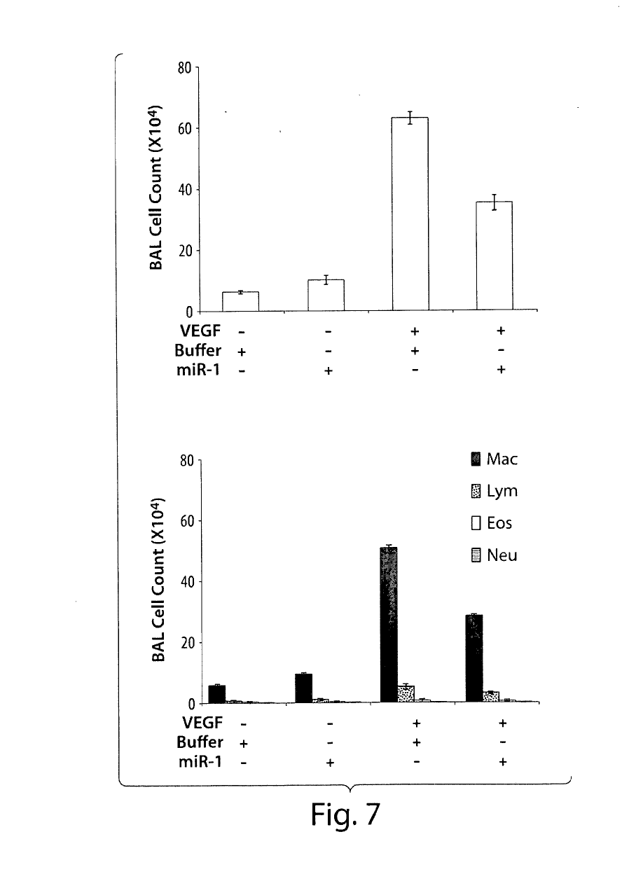

The data demonstrate that BAL fluid collected from VEGF₁₆₅ transgene (+) mice contains more inflammatory cells than the BAL fluid collected from VEGF₁₆₅ transgene (−) mice (FIG. 7). MiR-1 supplementation decreases the inflammatory cell count in both VEGF₁₆₅ transgene (+) and VEGF₁₆₅ transgene (−) mice compared to supplementation with buffer alone (FIG. 7). With respect to particular cell types, the data show that macrophage cells are particularly over abundant in the VEGF₁₆₅ transgene (+) mouse BAL fluid. As such, the effect of miR-1 supplementation is most prominently observed with the macrophage cell type, however, the data suggest that the relative abundances of all cell types are deceased following treatment with miR-1 (FIG. 7, right panel).

### Example 9

### MiR-1 Supplementation Decreases Angiogenesis in the Trachea of VEGF₁₆₅ Transgenic Mice

The presence of angiogenesis was determined following collection of trachea tissue from VEGF₁₆₅ transgene (−), and VEGF₁₆₅ transgene (+) mice supplemented with MiR-1 or buffer as described in Example 7.

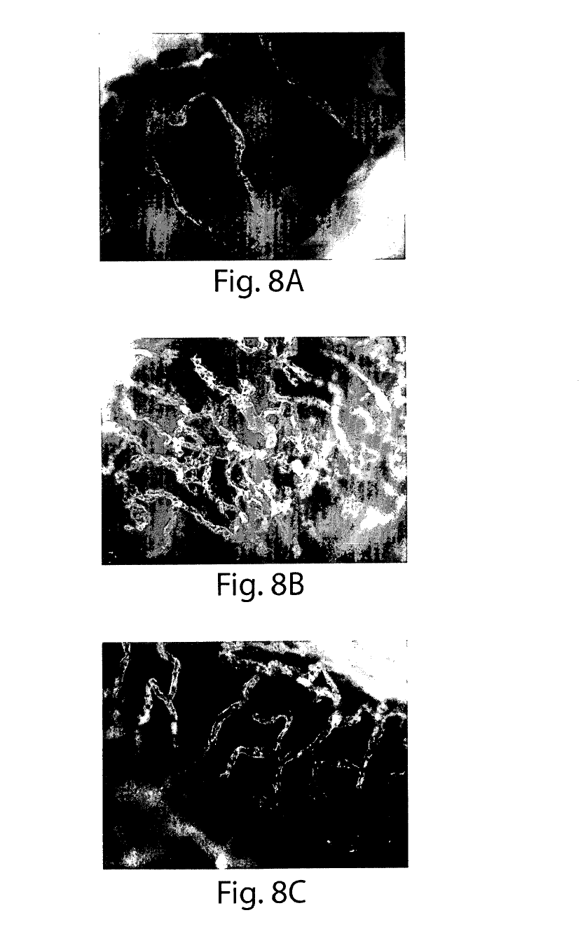

FIG. 8 shows a series of photographs of mouse trachea tissue collected from (A) VEGF₁₆₅ transgene (−), (B) and VEGF₁₆₅ transgene (+) mice supplemented with buffer and (C) VEGF₁₆₅ transgene (+) mice supplemented with miR-1. Mouse trachea prepared and stained with anti-CD31 antibody to visualize endothelial cells as shown previously (Lee et al. Nature Med. 2004 October: 10(10): 1095-103).

The data illustrate that angiogenesis occurs in the VEGF₁₆₅ transgene (+), but not the wild type (WT, VEGF₁₆₅ transgene (−)) trachea tissue. Importantly, miR-1 supplementation abrogates VEGF-induced angiogenesis.

### Example 10

### MiR-1 Inhibits the Proliferative Effect of VEGF in Cell Culture

MLECs were seeded at 1×10⁵ per well in 6-well plates and transfected with 100 picomoles of either miR-1 or QS (double stranded negative control RNA) diluted in Optimem®1 (Gibco) using 5 μl of Lipofectamin™ 2000 (Invitrogen) transfection reagent as described in the instruction manual. Medium was changed after ˜12 hours and cells were kept under complete medium (DMEM/F12, 20% FCS) for an additional 24 hours. These cells were then starved for 12 hours under starvation medium (DMEM/F12 only) and stimulated with 150 ng/ml of recombinant human VEGF for 24 hours. The number of viable cells in each well was counted using a hemocytometer after 5 minute incubation with 0.4% Trypan Blue stain (Gibco). Each experiment was done in n=6 replicates.

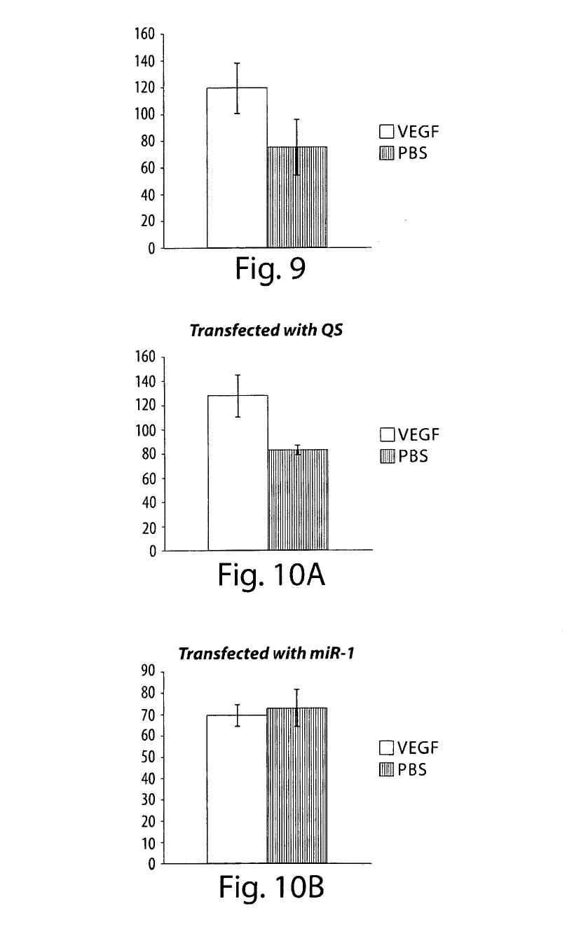

VEGF induces a proliferative response in MLECs in culture. As shown in FIG. 9, the cell number increases by 25-40% after 24 hour stimulation with VEGF (as compared to PBS). MLECs were transfected with either miR-1 or a negative control double stranded RNA (QS) and stimulated with VEGF 48 hours after transfection. As shown in FIG. 10, transfection with miR-1 (FIG. 10B), and not with the negative control (FIG. 10A), inhibits the VEGF-induced proliferation.

The effect of miR-1 upon endothelial proliferation has implications for angiogenesis, a process in which vascular endothelial cells must proliferate in order for blood vessels and capillaries to either grow or remodel. As such, the ability of miR-1 to decrease endothelial cell proliferation is one mechanism by which miR-1 decreases VEGF-mediated angiogenesis. In additional aspects of the invention, angiogenesis is an important factor in the progression and increasing severity of cancer. In this light, the ability of miR-1 to decrease endothelial cell proliferation is a mechanism by which miR-1 decreases angiogenesis which, in turn, decreases the severity of cancer and treats cancer. Moreover, vascular remodeling is a common occurrence in inflammatory disorders. As such the ability of miR-1 to decrease endothelial cell proliferation is a mechanism by which miR-1 decreases vascular remodeling, and in turn, decreases inflammation or treats an inflammatory disorder.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for treating a disorder caused by the ectopic or overproduction of a vascular endothelial growth factor (VEGF) polypeptide by at least one cell in a subject comprising administering to said subject an effective amount of an miRNA composition to decrease at least one activity of a VEGF polypeptide on said cell.

2. A method for treating a disorder caused by the ectopic or overproduction of a vascular endothelial growth factor (VEGF) polypeptide by at least one cell in a subject comprising administering to said subject an effective amount of an miRNA inhibitor composition to decrease at least one activity of a VEGF polypeptide on said cell.

3. A method for enhancing wound healing by decreasing production of a vascular endothelial growth factor (VEGF) polypeptide by at least one cell in a subject comprising administering to said subject an effective amount of an miRNA composition to decrease at least one activity of a VEGF polypeptide on said cell, thereby decreasing the occurrence of VEGF-mediated prolonged or abortive wound healing.

4. A method of decreasing angiogenesis in a tissue, comprising administering to said tissue an effective amount of an miRNA composition to decrease at least one activity of a VEGF polypeptide on said tissue.

5. A method of decreasing inflammation in a tissue, comprising administering to said tissue an effective amount of an miRNA composition to decrease at least one activity of a VEGF polypeptide on said tissue.

6. A method of decreasing hemorrhage in a tissue, comprising administering to said tissue an effective amount of an miRNA composition to decrease at least one activity of a VEGF polypeptide on said tissue.

7. A method of decreasing endothelial cell proliferation in a tissue, comprising administering to said tissue an effective amount of an miRNA composition to decrease at least one activity of a VEGF polypeptide on said tissue.

8. A method of increasing wound healing in a tissue, comprising administering to said tissue an effective amount of an miRNA composition to decrease at least one activity of a VEGF polypeptide on said tissue.

9. The method of any one of claims 1-8, wherein the method further comprises determining the activity of a VEGF polypeptide.

10. The method of any one of claims 1-8, wherein the method further comprises comparing the activity of a VEGF polypeptide prior to administration of said composition to the activity of a VEGF polypeptide following administration of said composition, wherein a change in said activity indicates that the subject is treated.

11. The method of claim 1 or 2, wherein said disorder is a cancer.

12. The method of claim 11, wherein said cancer is a solid tumor selected from the group consisting of adrenocortical carcinoma, AIDS-related cancers, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, central nervous system lymphoma, cervical cancer, colon cancer, colorectal cancer, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, hypopharyngeal cancer, intraocular melanoma, islet cell tumors (endocrine pancreas), Kaposi Sarcoma, kidney (renal cell) cancer, kidney cancer, laryngeal cancer, lip and oral cavity cancer, liver cancer, non-small cell lung cancer, small cell lung cancer, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine sarcoma, skin cancer (melanoma), merkel cell skin carcinoma, small intestine cancer, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilms Tumor.

13. The method of claim 1 or 2, wherein said disorder is angiogenesis.

14. The method of claim 13, wherein said angiogenesis is vasculogenesis.

15. The method of claim 1 or 2, wherein said disorder is a fibrotic disorder.

16. The method of claim 15, wherein said fibrotic disorder is injection fibrosis, endomyocardial fibrosis, mediastinal fibrosis, myelofibrosis, retroperiotoneal fibrosis, progressive massive fibrosis, nephrogenic systemic fibrosis, interstitial lung disease (ILD), idiopathic pulmonary fibrosis (IPF), scleroderma, radiation-induced pulmonary fibrosis, bleomycin lung, sarcoidosis, silicosis, pulmonary fibrosis, familial pulmonary fibrosis, nonspecific interstitial pneumonitis, autoimmune disease, renal graft transplant fibrosis, heart graft transplant fibrosis, liver graft transplant fibrosis, scarring, glomerulonephritis, cirrhosis of the liver, systemic sclerosis, or proliferative vitreoretinopathy.

17. The method of claim 1 or 2, wherein said disorder is wet age-related macular degeneration (wet AMD).

18. The method of claim 1 or 2, wherein said disorder is an inflammatory disorder.

19. The method of claim 18, wherein said inflammatory disorder is asthma, interstitial lung disease, chronic bronchitis, eosinophilic bronchitis, eosinophilic pneumonia, pneumonia, atopic dermatitis, atopy, allergic rhinitis, idiopathic pulmonary fibrosis, scleroderma, emphysema, autoimmune diseases, chronic prostatitis, glomerulonephritis, hypersensitivities, inflammatory bowel diseases, pelvic inflammatory disease, reperfusion injury, rheumatoid arthritis, transplant rejection, vasculitis, allergies, myopathies, or chronic obstructive lung disease.

20. The method of any one of claims 1-8, wherein said miRNA composition comprises miR-1, or any homolog thereof.

21. The method of any one of claims 1-8, wherein said miRNA composition comprises miR-203, or any homolog thereof.

22. The method of any one of claims 1-8, wherein said miRNA composition comprises miR-21, or any homolog thereof.

23. The method of any one of claims 1-8, wherein said miRNA composition comprises miR-468, miR-1, miR-451, miR-706, miR-486, miR-203, miR-494, miR-714, miR-705, miR-21, or any combination or any homolog thereof.

24. The method of any one of claims 1-8, wherein the composition comprises a pharmaceutically acceptable carrier.

25. The method of any one of claims 1-8, wherein the composition is administered systemically.

26. The method of any one of claims 1-8, wherein the composition is administered locally.

27. The method of any one of claims 1-8, wherein the VEGF polypeptide is VEGFA, VEGF-B, VEGF-C, VEGF-D, or PGF.

28. The method of any one of claims 1-8, wherein the VEGF polypeptide is an isoform of VEGFA.

29. The method of any one of claims 1-8, wherein the VEGF polypeptide is human.
